(19) United States
(12) Patent Application Publication
(10) Pub. No.: US 2005/0153304 A1
(43) Pub. Date: Jul. 14, 2005

(54) Multivariate profiling of complex biological regulatory pathways

(75) Inventors: Kevin Gardner, Montgomery Village, MD (US); Wendy Jo Freebern, Cazenovia, NY (US)

Correspondence Address: VENABLE LLP, P.O. BOX 34385, WASHINGTON, DC, 20435-9998, US

(73) Assignee: Government of the USA, as represented by the secretary, Department of Health and Human Services, Rockville, MD (US)

(21) Appl. No.: 10/822,140

(22) Filed: Apr. 12, 2004

Related U.S. Application Data
(60) Provisional application No. 60/461,410, filed on Apr. 10, 2003.

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

The present invention relates, e.g., to a method for generating and analyzing multi-factorial biological response profiles, comprising (a) exposing each member of a plurality of expression control sequences, each of which is operatively linked to a heterologous reporter sequence, independently, to at least about three stimuli from a first set of stimuli, wherein at least about two of the stimuli in said first set of stimuli are, optionally, combined in an intra-set combinatorial fashion; (b) detecting a first category of responses of said expression control sequences to said stimuli; and (c) generating a response profile for each of said expression control sequences. The method may further comprise (d) exposing each of said members of the plurality of expression control sequences, independently, to one or more additional sets of stimuli, optionally wherein at least about two of the stimuli in each of said additional sets of stimuli are combined in an intra-set combinatorial fashion, in an inter-set combinatorial fashion with set first set of stimuli; (e) detecting the first category of responses of said expression control sequences to the stimuli in d); and (f) generating a response profile for each of said expression control sequences, which includes the responses detected in b) and in e). Raw response profiles are preferably analyzed by multivariate statistical methods, using a computer.

### FIELD OF THE INVENTION

This invention relates generally to the use of bioinformatics to analyze complex biological systems.

### BACKGROUND INFORMATION

Studies of gene expression can provide insight into gene function and can aid in the discovery new methods of treatment for a variety of conditions, including genetically related diseases. However, few methods exist to interpret the massive amount of information resulting from the expression of many genes simultaneously, especially when the genes that are being expressed are subject to several variables, conditions and/or parameters. The present invention relates, e.g., to methods, using high throughput procedures and bioinformatics technology, for obtaining and analyzing large sets of responses of expression control sequences, such as promoters (or other biological entities), to multiple stimuli. The information obtained can be useful, for example, for developing or identifying drug targets and therapies, and for the dissection of complex regulatory pathways in a cell.

### BRIEF DESCRIPTION OF THE DRAWINGS

FIG. 1 shows an analysis of regulatory elements that govern T-cell activation in the presence of T-cell mitogens, in the presence and absence of immunomodulatory drugs.

Plotted is the activation profile of 16 different combinations of mitogens (see table one) in the absence of immunomodulatory drugs (red), the presence of 100 nM cyclosporine A (yellow) and with 10 uM SB203589, an anti-inflammatory mitogen activated kinase inhibitor (blue).

FIG. 2 shows an analysis by hierarchical clustering.

The data from FIG. 1 were re-analyzed by hierarchical clustering. On the left is shown a heat diagram that indicates the clustering of the activation profiles of the mitogen and drug combinations (rows) according to how they activate the eight different promoter elements or “transcriptional targets” (columns). The color code indicates the % maximum fold activation ×100. On the right is a dendrogram that represents the similarities and differences between the promoter elements based on how they responded to the mitogen/drug combinations as illustrated on the right.

FIG. 3 shows an analysis by non-supervised hierarchical clustering.

This analysis distinguishes and groups transcriptional targets by the drug sensitivity of mitogen response profiles. Analysis of the data in FIG. 3 by self-organizing maps (SOM) separates the 8 promoter elements into 4 distinct classes. (Top) Each corner of the square indicates a separate class containing promoter elements that respond similarly. The size of the circles indicate the size of the class. (Bottom) The bottom plot shows an average profile or “centroid” for each class.

FIG. 4 shows Principal Components Analysis (PCA) of multiple drug profiles.

The conditions outlined in Table I were analyzed by principal components analysis, which reveals major outliers in the presence of IGF-1 (the four blue data points, individually labeled).

FIG. 5 shows Principal Component Analysis of vector differences between mitogen profiles in the absence and presence of drug.

This analysis reveals significant contrasts and similarities of drug targeting. The mitogen activation profiles under control conditions shown in red FIG. 4 were used to calculate subtraction vectors for each profile in the presence of a drug treatment. These vectors were then analyzed by PCA to show similarities and differences in targeting.

FIG. 6 shows a “widescreen” analysis of transcriptional activation profiles from high throughput transfections.

This analysis reveals clear patterns of drug, mitogen and hormone action. FIG. 6 shows a heat diagram from 3072 transfection under the conditions indicated. Sixteen different combinations of mitogens (see table one) were assayed for their ability to stimulate 8 promoter elements (columns) in the presence and absence of different immuno-modulatory agents. The data are represented in % maximum activation ×100. Color code on the right indicates 0% to 170% in a color gradient from blue to red. (Pink indicating 100% maximal stimulation in the absence of modifying agents).

FIG. 7 shows profiling of transcriptional targets by nuclear run-on analysis using miniaturized nucleic acid arrays.

Shown is a nuclear run-on hybridization of ³²P labeled nascent RNA generated by nuclei isolated from human T-cells stimulated 45, 90 or 180 min with ionomycin and phorbol ester. Anti-sense oligonucleotides from four genes, IL-2, p21, 14-3-3 sigma, and GAPDH were spotted by hand onto glass slides, each spot representing an increment of 200 base pairs beginning at −200 base from the start of transcription.

FIGS. 8A and 8B show an analysis of mitogen profiles in “proteomic space”

FIG. 8A shows a PCA analysis of the manner in which the mitogen profile of the levels of phospho-retinoblastoma protein, phospho-AKT kinase, PCNA protein, phospho-jun protein, and total phospho-tyrosine levels are influenced in T-cells in the presence of cyclosporine A (control profile in red, 100 nM cyclosporine treated in green). FIG. 8B shows a PCA analysis of how the profile changes with treatment in the presence of 10 nM TGFbeta (control in red, TGF beta in green).

### DESCRIPTION OF THE INVENTION

The present invention relates, e.g., to a method for generating and analyzing multi-factorial biological response profiles (including expression profiles), comprising
a) exposing each member of a plurality of expression control sequences, each of which is operatively linked to a heterologous reporter sequence, independently, to at least about three stimuli from a first set of stimuli, wherein at least about two (e.g., at least about three) of the stimuli in said first set of stimuli are, optionally, combined in an intra-set combinatorial fashion,
b) detecting a first category of responses of said expression control sequences to said stimuli, and
c) generating a response profile for each of said expression control sequences.

The method may further comprise
d) exposing each of said members of the plurality of expression control sequences, independently, to one or more additional sets (e.g., a second, third, fourth, fifth etc. set, preferably to one additional set) of stimuli, optionally wherein at least about two (e.g., at least about three) of the stimuli in each of said additional sets of stimuli are combined in an intra-set combinatorial fashion,
in an inter-set combinatorial fashion with set first set of stimuli,
e) detecting the first category of responses of said expression control sequences to the stimuli in d), and
f) generating a response profile for each of said expression control sequences, which includes the responses detected in b) and in e).

As used herein, the term “expression control sequence” means a polynucleotide sequence that regulates expression of a polypeptide coded for by a polynucleotide to which it is functionally (“operably”) linked. Expression can be regulated at the level of the mRNA or polypeptide. Thus, the term expression control sequence includes mRNA-related elements and protein-related elements. Such elements include promoters, domains within promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter.

In a preferred embodiment, each of the expression control sequences of interest is cloned in a recombinant construct, so that the expression control sequence is operatively linked to a reporter sequence (a heterologous reporter). Methods of making recombinant constructs are conventional. Such methods, as well as many of the other molecular biological methods used in conjunction with the present invention, are discussed, e.g., in Sambrook, et al. (1989), Molecular Cloning, a Laboratory Manual, Cold Harbor Laboratory Press, Cold Spring Harbor, N.Y.; Ausubel et al. (1995). Current Protocols in Molecular Biology, N.Y., John Wiley & Sons; Davis et al. (1986), Basic Methods in Molecular Biology, Elseveir Sciences Publishing, Inc., New York; Hames et al. (1985), Nucleic Acid Hybridization, IL Press; Dracopoli et al. Current Protocols in Human Genetics, John Wiley & Sons, Inc.; and Coligan et al. Current Protocols in Protein Science, John Wiley & Sons, Inc.

Suitable reporter sequences will be evident to those of skill in the art. The reporter sequence can be a polynucleotide, which is detected by, e.g., specific hybridization procedures. These procedures are conventional and well known to the skilled worker. Alternatively, the reporter sequence may encode a protein whose presence and/or activity is measured. The amount and/or activity of the reporter protein serves as an indirect measure of gene expression regulated by the expression control sequence (e.g., mRNA initiating at a promoter sequence, or protein translated from the mRNA into protein). Any of a variety of conventional reporter proteins can be employed, including, e.g., green fluorescent protein, luciferase, β-galactosidase, chloramphenicol acetyltransferase, or the like. The use of the reporter protein, luciferase (Luc), is illustrated in Example I. Techniques to detect the amount and/or activity of protein reporters are conventional. Some typical techniques are discussed elsewhere herein.

In a preferred embodiment, the expression control sequences assayed by the methods of the invention are involved in similar (e.g., coordinate) pathways. For example, they may exhibit similar expression, or, when expressed under the same conditions, are likely to act together or are involved in similar processes. Thus, for example, the methods of the invention allow the analysis of expression control sequences that are important for regulating a particular pathway of interest, and may allow one to analyze a subset of genes that react in a particular way to the agents, or that are co-regulated. Examples of coordinate biological activity include, e.g., apoptosis, DNA repair, angiogenesis, signal transduction, vascular invasion, cell growth, reproduction, division, motility, differentiation, activation, differentiation, or other cellular responses, any of which can be studied in any cell type of interest. Other coordinate activites are T-cell activation, neurogenesis or nerve regeneration, and myogenesis or muscle regeneration.

In a particularly preferred embodiment, which is illustrated in the examples herein, the expression control sequences are from genes that play a role in signal transduction pathways (such as the pathways related to T-cell activation) and/or are from genes that are affected by (responsive to) signal transduction events. Each of the expression control sequences is introduced (e.g., electroporated) into a suitable cell, which is, for example, in a well of a multi-well microtiter plate. A wide variety of suitable cells will be evident to the skilled worker, including many established cell lines. Suitable cells will comprise one or more genes that encode signal transduction proteins, and/or one or more genes that are responsive to signal transduction proteins. In a preferred embodiment, T-lymphocytes or related cells are used, including primary T-cells (e.g., cells from leukemic patients), and cells from appropriate cell lines, such as Jurkat cells. Other cells that can be used include other types of immune cells, such as B-cells, dendritic cells, plasma cells, eosinophils, mast cells and basophils. Tumor cells, from any type of tumor, are also suitable host cells. Preferably, primary cells used in methods of the invention grow naturally in suspension (e.g., cells from the blood), although any cell line can be used, regardless of whether the cells grow in suspension. Among the cells that can be used are cells from transgenic animals (e.g., transgenic mice) with specific genetic characteristics of interest; preferably, lymphocytes from such animals, which are easy to assay, are used.

Endogenous gene products, including those involved in signal transduction pathways, will interact with the expression control sequences present in the constructs. By monitoring the expression of the reporter sequences, an investigator can determine the degree of activity of each expression control sequence in this cellular context. When the expression control sequences are exposed to one or more stimuli, an investigator can further assess the effect of the stimuli on the activity of the expression control sequence, in this cellular context. One of skill in the art can readily select an appropriate cell and appropriate expression control sequences for the characterization of a particular cellular pathway of interest.

To study phenomena like apoptosis, angiogenesis, or cell motility, which occur in all cell types, one may use any convenient cell as a host for an appropriate set of expression control sequences, which will be evident to the skilled worker. Fibroblasts are particularly well suited to this type of experiment. To study neurogenesis or nerve regeneration, appropriate expression control sequences will be introduced into nerve cells. To study stem cell differentiation, appropriate expression control sequences can be introduced into stem cells, and the effects of various agents can be tested to identify which promoters are turned on during specific types of cell differentiation. In many cases, it will be of interest to compare expression profiles elicited in response to a given set of stimuli in two related cell types. For example, one can compare senescent cells to non-senescent cells, in order to identify factors that are important for reactivating senescent cells.

In a preferred embodiment, samples are taken from patients, and are studied by the methods of the invention, for example to characterize properties of the disease. For example, patients suffering from leukemia have populations of normal and diseased cells in their blood. By comparing the responses of normal and diseased cells (e.g., mononuclear cells from blood or bone marrow) to a battery of stimuli, one can evaluate how the diseased cells react compared to the normal ones, which can allow the identification of metabolic or regulatory pathways that have been disrupted in the diseased cells. Similarly, one can compare the responses of tumor and related non-tumor cells. Also, by comparing the expression patterns of cells that are resistant or sensitive to a drug of interest, when challenged with the drug, one can identify whether a given cell (for example, from a patient) will be resistant to the drug. In another embodiment, one can compare samples from closely related family members, such as siblings, who have been exposed to different environmental conditions (e.g., pollutants or suspected carcinogens), to evaluate the physiological effects of those conditions. Many other uses of the inventive method will be evident to the skilled worker.

Many art-recognized methods are available for introducing polynucleotides, such as the constructs of the invention, into cells. The conventional methods that can be employed, include, e.g., transfection (e.g., mediated by DEAE-Dextran or calcium phosphate precipitation), infection via a viral vector (e.g., retrovirus, adenovirus, adeno-associated virus, lentivirus, pseudotyped retrovirus or poxvirus vectors), injection, electroporation, sonoporation, a gene gun, liposome delivery (e.g., Lipofectin®, Lipofectamine®(GIBCO-BRL, Inc., Gaithersburg, Md.), Superfect® (Qiagen, Inc. Hilden, Germany) and Transfectam® (Promega Biotec, Inc., Madison, Wis.), or other liposomes developed according to procedures standard in the art), or receptor-mediated uptake and other endocytosis mechanisms.

In a most preferred embodiment, constructs are electroporated into the cells, preferably in a multi-well microtiter dish, such as a 96-well microtiter dish. In this manner, about 96 constructs, for example, can be simultaneously and efficiently introduced into (electroporated into) cells. A suitable device for performing such electroporations is a device sold by BTX Instrument Division, Harvard Apparatus Inc., 84 October Hill Road, Holliston, Mass. 01746-1388.

In some embodiments, one can assess the response of expression control sequences to stimuli in the context of chromatin. For example, episomal vectors bearing expression control sequences of interest can be introduced into recipient cells and allowed to form a chromatin structure identical to that of the endogenous genome (e.g., to become coated with a complete complement of histone and non-histone proteins). This can be accomplished, for example, by allowing the cell to undergo one cell cycle in the presence of the episomal vector. The cell can then be exposed to stimuli by the methods of the invention. Alternatively, naturally occurring expression control sequences may be studied.

In general, methods of the invention involve the simultaneous analysis of a plurality of expression control sequences. For example, at least about 2, about 5, about 10, about 20, about 50, about 100, about 500, about 10³, about 10⁴ or more biological entities can be analyzed simultaneously.

Often, it is desirable to include biological entities that can serve as positive or negative controls, or for purposes of normalization of the data. Positive controls include, but are not limited to, viral expression control sequences that are known to be highly active in human cells, including promoter sequences from CMV, RSV, HTLV-I and HTLV-III. In addition the viral sequences may also be used as test or reference sequences for the evaluation of anti-viral drugs that target viral genes and gene products that depend on human cellular transcriptional machinery to complete the intra-cellular life cycle. This could be extended to all known viral pathogens that replicate inside human cells and use the host transcriptional machinery. Negative expression control sequences include, e.g., minimal basal promoters which contain only nucleotide sequences that encompass the TATA box core promoter sequence.

A variety of types of “stimuli” can interact with expression control sequences, directly or indirectly, thus inducing or altering cellular responses, and moving the system away from stasis or equilibrium. Such stimuli can be evaluated by the methods of the invention. In general, these stimuli fall into two broad classes: environmental stimuli, such as heat, light, radiation or changes in oxygen concentration, and physical agents, such as biological agents, chemical compounds, drugs, putative drugs, agents known to regulate expression control sequences of interest, toxins, and the like. In the methods of the present invention, a plurality of expression control sequences can be exposed to, for example, more than one physical agent, more than one environmental stimulus (e.g., a change in an environmental parameter), or a combination of physical and environmental stimuli.

Physical agents include, e.g., chemical compounds, biological agents, drugs, drug candidates (putative drugs), toxins, modulatory agents (e.g., agents that stimulate or inhibit a reaction), or the like. Of particular interest are agents that affect cellular activity, including, e.g., DNA damaging agents; oxidative stress-inducing agents; pH-altering agents; membrane-disrupting agents; metabolic blocking agents; chemical inhibitors; ligands for cell surface receptors; antibodies; transcription promoters, enhancers, or inhibitors; translation promoters or inhibitors; protein-stabilizing agents; protein destabilizing agents; stimulatory and/or regulatory agents, such as mitogens, growth factors or hormones; or combinations thereof. For studies of transcriptional regulation, agents can be used which interact directly with a regulatory element, or which act indirectly (e.g., they act upstream or downstream from the element, affecting the secondary or tertiary structure of the DNA or mRNA, or they interact with other proteins in a transcription complex).

In one embodiment, an agent that is a protein or peptide can be introduced into a cell as a polynucleotide that comprises an expression control sequence operatively linked to a coding sequence for the peptide or protein. The protein or peptide is then produced within the cell. For example, polypeptides having a known effect on genes of interest can be introduced. One such polypeptide is p53, which has been shown to inhibit expression of the IL-2 promoter.

Examples of DNA damaging agents include, e.g., intercalation agents such as ethidium bromide and alkylating agents such as methyl methanesulfonate. Examples of membrane disrupters include, e.g., Triton X-100, sodium dodecyl sulfate (SDS), and various detergents. Examples of metabolic blocking and/or energy blocking agents include, e.g., azidothymidine (AZT), ion (e.g. Ca⁺⁺, K⁺, Na⁺) channel blockers, α and β adrenoreceptor blockers, histamine blockers, and the like. Examples of chemical inhibitors include, e.g., receptor antagonists and inhibitory metabolites/catabolites (for example, mavelonate, which is a product of and in turn inhibits HMG-CoA reductase activity).

Examples of cell surface receptor ligands include, but are not limited to, various hormones (estrogen, testosterone, other steroids), growth factors, and G-protein-coupled receptor ligands. See, e.g., the ligands listed in Table 1 herein. Examples of antibodies include, e.g., antibodies directed against TNFα, TRAIL, or the HER2 growth factor receptor, or which are specific for any of the participants in signal transduction pathways.

Examples of oligonucleotides include, e.g., ribozymes, anti-sense oligonucleotides, and RNAi molecules.

Ribozymes are RNA molecules that have an enzymatic or catalytic activity against sequence-specific RNA molecules (see, for example, Intracellular Ribozyme Applications: Principles and Protocols, J. Rossi and L. Couture, eds. (1999, Horizon Scientific Press, Norfolk, UK)). Ribozymes can be generated against any number of RNA sequences, as shown in the literature for a number of target mRNAs, including calretinin, TNFα, HIV-1 integrase, and the human interleukins.

Antisense oligonucleotides can be used to control gene expression through methods based on binding of a polynucleotide to DNA or RNA. Without wishing to be bound to any particular mechanism, types of antisense oligonucleotides and proposed mechanisms by which they function include, e.g., the following: The 5′ coding portion of a polynucleotide sequence which encodes for a mature polypeptide of the present invention can be used to design an antisense oligonucleotide (e.g., an RNA, DNA, PNA etc. oligonucleotide) of any site which is compatible with the invention, e.g., of from about 10 to 40 base pairs in length. The antisense oligonucleotide can hybridize to the mRNA and block translation of the mRNA molecule into a polypeptide (see e.g., Okano, J. Neurochem., 56:560 (1991); Oligodeoxynucleotides as Antisense Inhibitors of Gene Expression, CRC Press, Boca Raton, Fla. (1988)). Alternatively, an oligonucleotide can be designed to be complementary to a region of the gene involved in transcription (see, e.g., Lee et al., Nucl. Acids Res., 6:3073 (1979); Cooney et al, Science, 241:456 (1988); and Dervan et al., Science, 251: 1360 (1991)), thereby preventing transcription and the production of encoded polypeptides. For further guidance on administering and designing antisense, see, e.g., U.S. Pat. Nos. 6,200,960, 6,200,807, 6,197,584, 6,190,869, 6,190,661, 6,187,587, 6,168,950, 6,153,595, 6,150,162, 6,133,246, 6,117,847, 6,096,722, 6,087,343, 6,040,296, 6,005,095, 5,998,383, 5,994,230, 5,891,725, 5,885,970, and 5,840,708.

RNAi molecules can also be used to inhibit gene expression, using conventional procedures. Typical methods to make and use interfering RNA molecules are described, e.g., in U.S. Pat. No. 6,506,559.

One embodiment of the invention is a method of comparing the efficacy of two different types of RNA inhibitors, such as antisense RNA vs. RNAi. By comparing the responses of a cell to the two different types of inhibitors, one can assess which class of inhibitors is preferred for inhibiting a group of genes or processes of interest, e.g., for use in a clinical setting.

Another class of agents that can be used are “small molecules,” sometimes referred to herein as “compounds,” which are isolated from natural sources or developed synthetically, e.g., by combinatorial chemistry. In general, such molecules may be identified from large libraries of natural products or synthetic (or semi-synthetic) extracts or chemical libraries according to methods known in the art. Those skilled in the field of drug discovery and development, for example, will understand that the precise source of test extracts or compounds is not critical to the methods of the invention. Accordingly, virtually any number of chemical extracts or compounds can be used in the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.).

Alternatively, libraries of natural compounds in the form of bacterial, fungal, plant, and animal extracts are commercially available from a number of sources, e.g., Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

Another class of stimuli is naturally occurring or synthetic (preferably, naturally occurring) agents that are known or expected to have an effect on an expression control sequence of interest, such as an expression control sequence that regulates the expression of a signal transduction gene, e.g., a signal transduction gene involved in T-cell activation. Suitable agents for studying expression control sequences of genes encoding signal transduction proteins include, e.g., stimulatory or regulatory agents, such as mitogens, growth factors, hormones, immunomodulatory agents, etc., and a variety of pharmacological agents that are known to modify cellular signaling pathways. Suitable agents of this category will be evident to those of skill in the art. Some such agents are listed in column 2 of Table 1. Others are discussed elsewhere herein.

Another category of stimuli comprises naturally occurring or synthetic agents that exhibit a pharmaceutical effect on patients, preferably on humans. Suitable agents of this category will be evident to those of skill in the art. Some such agents are listed in column 3 of Table 1. Others are discussed elsewhere herein.

Alternatively, the stimuli can take the form of environmental alterations that affect expression control sequences, such as, e.g., electromagnetic radiation, particle radiation, an electric or magnetic field or force, a mechanical field or force, pressure, light waves (e.g., UV or infrared), or changes in temperature, humidity, pH, oxygen concentration, or other growth or incubation conditions. Examples of oxidative stress agents include, e.g., hydrogen peroxide, superoxide radicals, hydroxyl free radicals, perhydroxyl radicals, peroxyl radicals, alkoxyl radicals, and the like. Examples of membrane disrupters include, e.g., the application of electric voltage potentials.

Physical agents to be evaluated can be introduced into cells by conventional methods. Small molecules, for example, can be added to the solution in which a cell resides and allowed to diffuse into the cell (e.g., be taken up by active or passive transport). Larger molecules may be introduced into a cell by conventional methods, including liposome-based methods, receptor-mediated endocytosis, etc. Transfection or electroporation can be used to introduce nucleic acids, such as antisense RNA, ribozymes, RNAi, or DNA constructs encoding a peptide or protein agent of interest, etc. Electroporation can also be used to introduce other large molecules, such as proteins, including antibodies, into a cell. If desired, a construct and at least one agent may be introduced into a cell together (e.g., co-transfected or co-electroporated).

In other cases, a physical agent may act on the surface of a cell, e.g., it may interact with a cell receptor. If desired, “stimuli” that serve as positive, negative or normalization controls can also be used. For example, inhibitors of transcription and translation can be used, including alpha amanitin, actinomycin D, or cyclohexide. Normalization controls in most cases are done by statistical replicates with normalization for protein concentrations of each lysate. Traditional normalization controls for transfection efficiency rely on promoter sequences that interfere and/or compete with the regulatory elements under study.

In embodiments of the invention, stimuli from a first set of stimuli, stimuli from a second set of stimuli, or both, may be combined in an “intra-set combinatorial fashion.” That is, one or more stimuli (e.g., at least about 2, or at least about 3) of at least one set may be combined with one another, for example pair-wise, three at a time, four at a time, etc. For example, if the first set includes stimuli A-1, A-2, A-3 and A-4, the following types of intra-set combinations of stimuli can be exposed to one of the biological entities: A-1 plus A-2; A-1 plus A-3; A-2 plus A-3; A-1 plus A-2 plus A-4; A-1 plus A-2 plus A-3 plus A-4, etc.

Often, it is desirable that substantially fewer than all of the possible intra-set combinations are used. The selection of suitable combinations may be made by one of skill in the art, on the basis of experimental results or information known to skilled workers. The selections will generally reflect the most biological significant or most informative of possible combinations. Selection of such a sub-set of combinations provides several advantages, e.g., it may render the method more efficient, render more likely the number of positive test results, etc.

In other embodiments of the invention, stimuli from a first set and second set (or still further sets) of stimuli are exposed to each member of a plurality of biological entities in an “inter-set combinatorial fashion.” That is, one or more stimuli of two sets are combined, for example pair-wise, three at a time, and so forth. For example, if the first set includes stimuli A-1, A-2, A-3 and A-4, and the second set includes B-1, B-2, B-3 and B-4, the following types of combinations of stimuli can be exposed to one of the biological entities: A-1 plus B-1; A-1 plus B-2; A-1 plus B-3; A-3 plus B-1; A-1 plus B-1 plus B-2; A-1 plus A-2 plus B-I plus B-2, etc.

In another embodiment of the invention, the stimuli in, e.g., a first set and a second set, represent different categories of stimuli. For example, when a set of stimuli comprises two different categories, the members of each category are non-overlapping, i.e., the two categories have no members in common. In some cases, members of the different categories may exhibit different properties. One of skill in the art will recognize stimuli that fall into different categories by virtue of exhibiting different properties.

Typical examples of physical agents that fall into such different categories include, for example:
a) agents that act at the surface of a cell (e.g., by interacting with a cell surface receptor) vs. agents that function within a cell,
b) agents that exhibit different mechanisms of action (e.g., agents that interact directly with a promoter element vs. agents that interact with a protein that is involved in transcription vs. agents that affect levels of translation or post-translational modification),
c) agents that have different chemical structures (e.g., agents that fall into different broad classes, such as polypeptides vs. polynucleotides vs. small molecule chemical compounds, etc.); or agents within a particular chemical class that differ from one another (e.g., peptides vs. full-length proteins; small molecule chemicals having different core structures; different types of RNAs, such as antisense RNA vs. RNAi vs. ribozymes, etc.),
d) agents that are produced within a cell (e.g., proteins that are “introduced” into a cell by transfection of a polynucleotide in which coding sequences for the agent are operatively linked to an expression control sequence) vs. agents that are introduced directly into a cell (e.g., a protein or peptide that is introduced into a cell),
e) agents having a known mechanism of action on a biological entity vs. agents not having a known mechanism,
f) agents having a known effect on the biological entity vs. test agents (e.g., putative drugs),
g) agents known to have an effect on at least one of the expression control sequences (e.g., mitogens, growth factors, hormones, or pharmacological agents that are known to interact with and/or modify cellular signaling pathways) vs. agents not known to have an effect on any of those expression control sequences.
h) naturally occurring agents (such as proteins or small molecules known to work in a cell in vivo) and artificially generated molecules (e.g., synthetic drugs, particularly those already in use in humans), and/or
i) physical agents vs. environmental stimuli,
j) or a combination thereof.

Environmental stimuli also fall into different categories. For example, exposure to heat clearly falls into a different category than exposure to UV irradiation, etc.

The stimuli within a given combination, whether combined in an inter-set or an intra-set combinatorial fashion, may be exposed to an expression control sequence together. That is, they may be added at substantially the same time to a well in a multi-well microtiter dish containing a cell that comprises an expression control sequence of interest. Alternatively, the stimuli within a given combination may be exposed consecutively to the expression control sequence, separated by a predetermined period of time. In a preferred embodiment, the stimuli are exposed to the expression control sequence together (at the same time, simultaneously).

In methods of the invention, a plurality of expression control sequences (in recombinant constructs), often a large number of such sequences, are exposed, independently, to many stimuli. Multi-pronged pipetters adapted to aliquot samples simultaneously to multi-well microtiter plates can facilitate the simultaneous additions of agents to the samples.

Any of a variety of responses that can be elicited by stimuli and detected according to the invention will be evident to the skilled worker. These responses may be mediated either by expression control sequences that have been introduced into a cell (exogenously introduced), or by endogenous expression control sequences in the cell. Responses to the stimuli also include indirect effects, such as the effects of proteins encoded by genes under the control of expression control sequences of interest (e.g., signal transduction proteins) on one or more other genes (e.g., other genes that are controlled by a signal transduction event, such as downstream genes in a signal transduction pathway). Other responses to stimuli may be elicited by mechanisms other than changes in expression.

As used herein, the term “responses of an expression control sequence” is meant to encompass responses mediated by the expression control sequence. That is, for example, in the presence of a stimulus a sequence may bind to a protein, which in turn exerts an effect, such as transcribing mRNA, etc. The sequence mediates the response to the stimulus, rather than responding directly to the stimulus, itself.

Detectable responses elicited by the stimuli include, e.g., changes in transcriptional initiation or elongation (e.g., levels of RNA produced in response to the stimulus), protein abundance or activity, stability, or transportation, compartmentalization, secretion, structural modification, or a combination thereof, of proteins, nucleic acids, or other cellular components (including lipids, such as lipids in membranes). Among the responses that can be measured following exposure to stimuli are, e.g., the movement of an RNA polymerase molecule along a DNA template (as determined by nuclear run-on analysis, e.g., as discussed in Example III), and the formation of protein-DNA complexes (as determined by kinetic analysis of protein-DNA complexes, e.g., as discussed in Example IV).

A response profile may include one or more of the responses discussed above. Responses that are not modulated by an exogenously introduced expression control sequence may be correlated, for example, with responses modulated by endogenous expression control sequences, and/or with responses that do not directly reflect a change in gene expression.

Responses may be measured at either a single time point or over a plurality of time points. Optionally, at least one measurement is collected prior to stimulation, as a control. Furthermore, responses may be measured as a function of the amount of a given stimulus, e.g., the concentration of a physical agent, or the degree of an environmental stimulus.

In one embodiment of the invention, a first category of responses of an expression control sequence to a battery of stimuli is detected (for example, mRNA transcription); and at least one further category of responses is also detected (e.g., expression of a particular protein of interest). For example, following the introduction of constructs of the invention into cells which are in wells of a microtiter plate, and exposure of the cells to stimuli as noted above, two or more aliquots may be removed from each well, and submitted to two or more different assays to detect two or more different categories of responses. In other words, the method comprises detecting two or more different categories of responses of the expression control sequences to the stimuli. The two or more categories of responses may then be combined to generate a response profile for each of the expression control sequences.

In some embodiments, assays are performed in parallel with assays involving exogenously introduced expression control sequences. For example, cells containing endogenous expression control sequences of interest may be studied in parallel (following exposure to the same battery of stimuli), using a nuclear run-on assay. Such sequences may be, e.g., naturally occurring endogenous sequences, or sequences that have been introduced into the cells by transfection and allowed to integrate into the genome of a host, thus providing a way to study the expression control sequences in the context of chromatin.

Among the categories of responses that can be detected are, e.g.,
a) levels of RNA produced in response to the stimuli,
b) levels of proteins translated from said RNA,
c) levels of post-translational protein modification,
d) movement of an RNA polymerase molecule along a DNA template (as determined by nuclear run-on analysis) in response to the stimuli, and/or
e) the formation of protein-DNA complexes (as determined by “kinetic promoter occupancy analysis” in response to the stimuli, and
f) changes in lipid membrane composition.

In some embodiments, cells that have been exposed to stimuli by methods of the invention are fractionated before the responses are detected. For example, nuclei can be gently extracted, and the endogenous response to stimuli (e.g., gene activation), is monitored with a high throughput nuclear run-on assay.

Any of a variety of conventional methods may be used to detect responses (e.g., expression levels) of biological entities to agents or changes in environmental conditions. These methods include, e.g., RNA transcription assays, protein expression assays, protein function assays, protein transportation/compartmentalization/secretion assay, phenotype-based cellular assays, metabolic assays, small molecule assays, ionic flux assays, reporter gene assays, membrane alteration/disruption assays, intercellular signaling assays, selective sensitivity-to-invasion assays, or other assays and analytical techniques known to one skilled in the art. The assay can be performed on the cells directly, or it can be performed on some derivative of the cells, such as cellular lysates, extracts, or separations.

Many of these methodologies and analytical techniques can be found in such references as Current Protocols in Molecular Biology, F. M. Ausubel et al., eds., (a joint venture between Greene Publishing Associates, Inc. and John Wiley & Sons, Inc., supplemented through 1999), Enzyme Immunoassay, Maggio, ed. (CRC Press, Boca Raton, 1980); Laboratory Techniques in Biochemistry and Molecular Biology, T. S. Work and E. Work, eds. (Elsevier Science Publishers B. V., Amsterdam, 1985); Principles and Practice of immunoassays, Price and Newman, eds. (Stockton Press, NY, 1991); and the like.

A useful detection assay is a nuclear run-on assay. Such an assay, as adapted to the high throughput/bioinformatics method of the invention, is described in Example II. Other useful assays include the proteomic analysis described in Example III, and the analysis of kinetic formation of protein-DNA complexes discussed in Example IV. Other useful assays include the following:

Changes in nucleic acid expression can be determined by polymerase chain reaction (PCR), ligase chain reaction (LCR), Qβ-replicase amplification, nucleic acid sequence based amplification (NASBA), and other transcription-mediated amplification techniques; differential display protocols; analysis of northern blots, enzyme linked assays, micro-arrays and the like. Examples of these techniques can be found in, for example, PCR Protocols A Guide to Methods and Applications (Innis et al. eds) Academic Press Inc. San Diego, Calif. (1990).

Alternatively, the expression pattern of genes can be rapidly analyzed as described by Wang et al. (Nucleic Acids Research (1999) vol. 27, pages 4609-4618). This technique employs PCR amplification of cDNAs that have been cleaved by frequently-cutting endonucleases, such as DpnII and NlaIII, and primed with defined sequences prior to amplification.

Another method for detecting molecular events within the plurality of cells utilizes real-time PCR, using, for example, molecular beacons or FRET (fluorescence resonance energy transfer). The FRET technique utilizes molecules having a combination of fluorescent labels which, when in proximity to one another, allows for the transfer of energy between labels (see, for example, X. Chen and P.-Y. Kwok, (1997) Nucleic Acid Research vol. 25, pp. 2347-2353).

Optionally, the responses of the plurality of cells can be monitored by fluorescence activated cell sorting, or FACS. A wide variety of flow-cytometry methods have been published. For a general overview of fluorescence activated flow cytometry see, for example, Abbas et al. (1991) Cellular and Molecular Immunology, W. B. Saunders Company; Coligan et al. (eds)(1991) Current Protocols in Immunology, and Supplements, John Wiley and Sons, Inc. (New York); and Kuby (1992) Immunology, W. H. Freeman and Company,. Fluorescence activated cell scanning and sorting devices are available from several companies, including, e.g., Becton Dickinson and Coulter.

Alternatively, high throughput screening systems utilizing microfluidic technologies, available, for example, from Agilent/Hewlett Packard (Palo Alto, Calif.) and Caliper Technologies Corp. (Mountain View, Calif.) could be employed for detecting the response(s) generated in the plurality of cell lines. The Caliper Lab Chip.™. technology uses microscale microfluidic techniques for performing analytical operations such as the separation, sizing, quantification and identification of nucleic acids (for further information, see www.calipertech.com).

In a preferred embodiment, the method of the invention is a high throughput method (e.g., is performed on a large number of samples, with a large number of variables, and at least some of the steps are performed automatically). In one embodiment of the invention, at least one of the processes is performed robotically.

A collection of data points representing the responses of one expression control sequence to a set of stimuli is termed herein a “raw response profile” for that expression control sequence. For example, a first category of responses, such as the degree of mRNA transcription, of each of a plurality of expression control sequences to each of several different combinations of stimuli may be detected (e.g., measured, or quantitated), to generate a raw response profile for each of the plurality of expression control sequences. Furthermore, as noted above, in one embodiment of the invention each member of the plurality of biological entities is further exposed, independently, to one or more further sets of stimuli (e.g., to a second, third, fourth etc. set of stimuli), and the response to the further stimuli (e.g., the degree of transcription) is detected. In such a case, the raw response profile reflects the responses of one of the expression control sequences to all of the sets of stimuli to which it is exposed. In another embodiment of the invention, more than one category of response to each stimulus is detected. For example, the degree of transcription and the degree of translation of a particular protein of interest in response to the set of stimuli may be also detected. In this case, the raw response profile reflects both types of responses to the set of stimuli.

The raw response profiles generated as above are, in general, highly complex and multi-dimensional. In a preferred embodiment, the responses are inputted into a computer and processed by multivariate statistical methods, such as, e.g., principal component analysis (PCA), hierarchical clustering, unsupervised neural networks and ANOVA studies. Such methods can reduce the number of dimensions in a raw response profile to a form that can be graphically displayed, e.g., two or three dimensions. Raw response profiles that have been processed by such multivariate statistical methods are sometimes called herein “processed response profiles.”

Observation of responses of expression control sequences as they occur over time and/or in response to one or more stimuli provides a dynamic view of the biomolecular activity of the cell.

For each experiment performed, the plurality of data points is gathered into a database and used to generate a raw response profile for the corresponding expression control sequence. The plurality of data points representing the responses to stimulation can be linear or nonlinear. In one embodiment of the invention, the generating the plurality of profiles consists of a) selecting a first expression control sequence from the plurality of expression control sequences; b) evaluating at least one response, and optionally multiple responses; c) recording the evaluation of the at least one response; and d) repeating these steps for additional expression control sequences in the plurality of expression control sequences. In another embodiment of the method of the invention, the evaluating and recording of information is performed on the entire plurality of expression control sequences simultaneously. During the recording step, the response (or responses) generated for each expression control sequence are entered into a raw profile database for further analysis. The entire set of expression control sequences can be evaluated for response to a stimulus, or a subset of the set of expression control sequences can be examined.

Generation of the plurality of raw profiles for the plurality of expression control sequences generally results in a large quantity of data, reflecting information related to the expression control sequences used and the responses measured for the plurality of expression control sequences. In one embodiment of the invention, the plurality of data points is entered as character strings, or as descriptors, into a database. The character strings or descriptors can be used to encode include any relevant information derived from or detected within the plurality of expression control sequences, including any physical characteristics, activities, or other information related to the cell types used and the responses detected. In general, the database is embodied in a computer or computer readable medium and can be accessed by a user and/or integrated system.

The raw response profiles can be analyzed by any of a variety of multivariate analytical means, such as multivariate analysis, n-dimensional space analysis, principal component analysis (PCA), difference analysis, n-dimensional space analysis, factor analysis, cluster analysis (including hierarchical clustering), and the like. In most preferred embodiments, the analytic tools used are principal component analysis, hierarchical clustering, unsupervised neural networks, ANOVA studies, or a combination thereof. The results can be used to generate a graphical representation of the collected data across a plurality of stimuli and/or a plurality of time points.

The information encoded in the database (i.e. the plurality of raw profiles) can be evaluated in the analyzing step of the method of the present invention. Analysis of the data involves the use of any of a number of statistical tools to evaluate the measured responses and changes based on type of change, direction of change, shape of the curve in the change, timing of the change and amplitude of change. This information can be used to perceive and interpret the impact that alterations, ranging from a “minor” change in a single nucleotide to major permutations in one or more metabolic pathway, can have on the biological systems network as a whole.

Multivariate statistics, such as principal components analysis (PCA), factor analysis, cluster analysis, n-dimensional analysis, difference analysis, multidimensional scaling, discriminant analysis, and correspondence analysis, can be employed to simultaneously examine multiple variables for one or more patterns of relationships (for a general review, see Chatfield and Collins, “Introduction to Multivariate Analysis,” published 1980 by Chapman and Hall, New York; and Hoskuldsson Agnar, “Predictions Methods in Science and Technology,” published 1996 by John Wiley and Sons, New York). Multivariate data analyses are used for a variety of applications involving these multiple factors, including quality control, process optimization, and formulation determinations. The analyses can be used to determine whether there are any trends in the data collected, whether the properties or responses measured are related to one another, and which properties are most relevant in a given context (for example, a disease state). Software for statistical analysis is commonly available, e.g., from Partek Inc. (St. Peters, Mo.; see www.partek.com).

One common method of multivariate analysis is principal component analysis (PCA, also known as a Karhunen-Loeve expansion or Eigen-XY analysis). PCA can be used to transform a large number of (possibly) correlated variables into a smaller number of uncorrelated variables, termed “principal components.” Multivariate analyses such as PCA are known to one of skill in the art, and can be found, for example, in Roweis and Saul (2000) Science 290:2323-2326 and Tenenbaum et al. (2000) Science 290:2319-2322.

For further information about multivariate statistics, see, e.g., www-binf.bio.uu.nl/BPA/ex22002.html (PCA); citeseer.nj.nec.com/329882.html (unsupervised neural networks); and the web site at physics.csbsju.edu/stats/anova.html (ANOVA). Hierarchical clustering is a basic computational method, which can be summarized as follows: Given a set of N items to be clustered, and an N×N distance (or similarity) matrix:
1. Start by assigning each item to its own cluster, so that if you have N items, you now have N clusters, each containing just one item. Let the distances (similarities) between the clusters equal the distances (similarities) between the items they contain.
2. Find the closest (most similar) pair of clusters and merge them into a single cluster, so that now you have one less cluster.
3. Compute distances (similarities) between the new cluster and each of the old clusters.
4. Repeat steps 2 and 3 until all items are clustered into a single cluster of size N.

The responses generated by a given plurality of expression control sequences can be grouped, or clustered, using multivariate statistics. Clusters for each different stimulation (treating) and observation (detecting) experiment are compared and a secondary set of correlations/noncorrelations are made. Based on these different sets of correlations, a network map can be created wherein the relative relationships of the different genetic elements can be established as well as how they may act in concert. In addition, the data can be visualized using graphical representations. Thus, for example, the temporal changes exhibited by the different biochemical and genetic elements within a genetically-related group of expression control sequences can be transformed into information reflecting the functioning of the expression control sequences within a given environment.

One aspect of the invention is a computer-implemented method for generating and analyzing multi-factorial biological response profiles, comprising
a) exposing each member of a plurality of expression control sequences, each of which is operatively linked to a heterologous expression control sequence, independently, to
at least about three stimuli from a first set of stimuli, wherein at least about two (e.g., at least about three) of the stimuli in said first set of stimuli are combined in an intra-set combinatorial fashion, and to
at least about three stimuli from a second set of stimuli, wherein at least about two of the stimuli in said second set of stimuli are optionally combined in an intra-set combinatorial fashion,
in an inter-set combinatorial fashion,
b) inputting into a computer responses of said expression control sequences to said stimuli, thereby generating a database which comprises a raw profile for each of the expression control sequences,
c) processing the data base comprising the raw profiles with
   i) principal component analysis,
   ii) hierarchical clustering,
   iii) unsupervised neural networks, and/or
   v) ANOVA studies,
   or a combination thereof, and, optionally,
d) displaying the processed profiles.

The present invention also provides an integrated system for deciphering the inter-relationships of expression control sequences (e.g., mechanisms of cellular function, or transcriptional targeting). The integrated system includes a plurality of expression control sequences, as discussed elsewhere herein. In this discussion of integrated systems, the term “cells” is often used. It is to be understood in this context that the “cells” referred to are generally cells into which have been introduced constructs bearing expression control sequences, operatively linked to reporters.

In addition, the integrated system has a detection system, which performs several functions. First, the detection system receives the plurality of cells (comprising constructs bearing expression control sequences). The detection system can accommodate whole cells, or a derivative thereof, for example, cell lysates or chromatographic eluents. Optionally, the detection system receives the plurality of cells in a multi-well container, such as a 96, 384, 768 or 1536 well plates (available from various suppliers such as VWR Scientific Products, West Chester, Pa.). The multi-well container can be a receptacle in which the exposing (e.g, treating or stimulating) event takes place. Additionally the multi-well container can accommodate further manipulations to the plurality of cells, such as generation of the cell line derivatives, preparation of isolated nuclei for nuclear run-on analysis, etc.

The detection system detects at least one response to one or more stimuli. The cells can be stimulated prior to insertion into the detection system, or after insertion. Detection of the at least one response can be achieved by a number of analytical techniques such as mass spectrometry; NMR spectroscopy; visible/UV/infra-red spectroscopy; fluorescence, phosphorescence, chemiluminescence and/or other types of photoemission spectroscopy (using either static or time-resolved methodologies); potentiometry, calorimetry; radiography; diffraction methodologies; and electron-pair resonance (EPR) spectroscopy, optionally coupled with techniques such as chromatography, electrophoresis (including capillary electrophoresis), microscopy, cytometry, and the like. Other detection methods are described elsewhere herein.

Additionally, the detection system generates a plurality of data points based upon both information related to the plurality of expression control sequences and the at least one response to the one or more stimuli. The data generated in general include data obtained from analysis of expression control sequences. However, particularly when other types of “biological entities” are studied (see below), the data can include other types of information as well. The data can include information related to cell type(s), gene sequences, genetic polymorphism, mRNA expression levels, mRNA splicing and/or modification events (such as polyadenylation, removal of leader sequences, and capping), transcript transportation events, mRNA expression ratios, protein expression levels, protein activity levels, protein modification levels, protein-protein interactions, reporter gene expressions/activities, protein transportation, localization and secretion events (including cross membrane and extracellular transport), cellular phenotypic alterations (including alterations in cell morphology), cellular properties (such as adhesion, nonadhesion, differentiation, invasion, proliferation, cell-cell interaction, synchronization, and termination), changes in cellular factors (including ionic and energy levels), and other observable changes that occur within cells.

Furthermore, the integrated system of the present invention has a data analyzing system in operational communication with the detection system. The data analyzing system comprises a computer or computer-readable medium having one or more logical instructions for organizing the plurality of data points into a database and one or more logical instructions for analyzing the plurality of data points. Optionally, the data analyzing system can also have one or more logical instructions for operating components of the detection system, and can be accessed by a user and/or the integrated system. The data analyzing system can be a computer running any available operating system (commercial or otherwise), or it can be another form of computational device known to one of skill in the art. Software for manipulating information descriptor elements is available, or can easily be constructed by one of skill using a standard programming language such as C, C++, Visual Basic, Fortran, Basic, Java, or the like. For example, a computer system can include software having descriptors of the data points, optionally modified for conjunction with a user interface (e.g., a GUI in a standard operating system such as a Windows, Macintosh, UNIX, LINUX, and the like), to manipulate the strings of characters or descriptors representing the plurality of profiles. Standard desktop applications including, but not limited to, word processing software (e.g., Microsoft Word™ or Corel WordPerfect™), spreadsheet and/or database software (e.g., Microsoft Excel™, Corel Quattro PrO™, Microsoft Access™, Paradox.™, Filemaker PrO™, Oracle™, Sybase™, and Informix™) can be adapted for generating, storing and/or analyzing the plurality of profiles.

The character strings or descriptors can be used to encode any relevant information derived from or detected within the plurality of cells, including any physical characteristics, activities, or other information related to the cells used and the responses detected. The logical instructions within the computer or computer-readable medium can optionally include software for performing, for example, multivariate analysis, principal component analysis, difference analysis, or n-dimensional space analysis. In addition, the integrated system can also provide an output file. The output file can be in the form of a graphical representation of part or all of the plurality of data points. Alternatively, the output file can comprise descriptors, for example, for entering this information into an alternative database or computer-readable medium.

Another aspect of the invention is a computer system for generating and analyzing multi-factorial biological response profiles, comprising
a) means for inputting responses into a database, wherein said responses are generated by
   i) exposing each member of a plurality of expression control sequences, each of which is operatively linked to a heterologous expression control sequence, independently, to
at least about three stimuli from a first set of stimuli, wherein at least about two (e.g., at least about three) of the stimuli in said first set of stimuli are combined in an intra-set combinatorial fashion, and to
at least about three stimuli from a second set of stimuli, wherein at least about two of the stimuli in said second set of stimuli are optionally combined in an intra-set combinatorial fashion,
in an inter-set combinatorial fashion, and
   ii) detecting the responses of said biological entities to said stimuli;
b) means for analyzing said inputted responses (e.g., means for reducing the number of dimensions of said inputted responses to three dimensions); and, optionally,
c) means for displaying the analyzed responses.

Another aspect of the invention is a computer-readable storage medium storing computer-readable program code for causing a computer to perform the following steps:
a) retrieving responses generated by
   i) exposing each member of a plurality of expression control sequences, each of which is operatively linked to a heterologous expression control sequence, independently, to
   at least about three stimuli from a first set of stimuli, wherein at least about two (e.g., at least about three) of the stimuli in said first set of stimuli are combined in an intra-set combinatorial fashion, and to
   at least about three stimuli from a second set of stimuli, wherein at least about two (e.g., at least about three) of the stimuli in said second set of stimuli are optionally combined in an intra-set combinatorial fashion, in an inter-set combinatorial fashion, and
   ii) detecting the responses of said biological entities to said stimuli,
b) processing the retrieved responses with a multivariate statistical method and, optionally,
c) displaying the processed responses.

The multivariate statistical method may be, e.g., principal component analysis, hierarchical clustering, unsupervised neural networks, and/or ANOVA studies, or a combination thereof.

Another aspect of the invention is a database (e.g., a reference database) of raw or processed response profiles, prepared by the method of the invention and having the corresponding inventive data structures. Such a database can be used, e.g., as a reference database for comparing the responses to an uncharacterized agent (e.g., a putative drug, uncharacterized herbal preparation, etc).

Another aspect of the invention is an integrated system for deciphering the inter-relationships of expression control sequences, or mechanisms of cellular function, comprising
a) a plurality of expression control sequences, each of which is operatively linked to a heterologous expression control sequence, which have been exposed, independently, to
   at least about three stimuli from a first set of stimuli, wherein at least about two (e.g., at least about three) of the stimuli in said first set of stimuli are combined in an intra-set combinatorial fashion, and to
   at least about three stimuli from a second set of stimuli, wherein at least about two (e.g., at least about three) of the stimuli in said second set of stimuli are optionally combined in an intra-set combinatorial fashion,
   in an inter-set combinatorial fashion,
b) a detection system for receiving the plurality of expression control sequences, wherein the detection system detects the responses of the expression control sequences to the stimuli, and generates a plurality of data points based upon the responses, and
c) a data analyzing system in operational communication with the detection system, the data analyzing system comprising a computer or computer-readable medium comprising one or more logical instructions for organizing the plurality of data points into a database and one or more logical instructions for analyzing the plurality of data points.

In other aspects of this integrated system, the detection system detects a signal or a result from an analytical technique; the analytical technique comprises an RNA transcription assay, a protein expression assay, a protein function assay, a phenotype-based cellular assay, a metabolic assay, a cofactor or small molecule assay, an ionic potential-measuring assay, a reporter gene assay, or a combination thereof; the detection system detects the at least one response (e.g., a plurality of responses) at a plurality of time points; the database comprises a plurality of profiles for the plurality of expression control sequences; the one or more logical instructions for analyzing the plurality of data points comprises software for generating a graphical representation of the plurality of responses and/or the plurality of time points; the one or more logical instructions for analyzing the plurality of data points comprises software for performing multivariate analysis for the plurality of data points, e.g., software for analyzing the plurality of data points in n-dimensional space, software for performing principal component analysis upon the plurality of data points, and/or software for performing difference analysis upon the plurality of data points; and the integrated system further comprising an output file, e.g., wherein the output file comprises a network model of the plurality of data.

The methods and devices of the invention have many uses, which will be evident to the skilled worker. For example, the methods may be adapted to be:
a) a method for determining the type of an unknown stimulus (e.g., drug or other agent, environmental effect, etc.);
b) a method for identifying/characterizing a modulatory or co-modulatory agent;
c) a method for identifying the cellular pathway affected by an agent (e.g., a growth factor etc. or a drug);
d) a method for determining whether a drug candidate has an activity similar to a known drug, (e.g., for determining whether a first agent and a second agent act on a cell(s) by a related mechanism of action, and/or exhibit a similar dose response);
e) a method for identifying a regulatory pathway, control point or therapeutic target;
f) a method for studying a combinatorial drug strategy in a pre-clinical setting;
g) a method for identifying a cell or organism that is sensitive or resistant to a drug composition;
h) a method for determining if a sample (e.g., from a patient) is susceptible to, or likely to benefit from, a particular treatment; or
i) a method for determining if an agent is toxic.

Such methods are based on comparisons of response profiles. A skilled worker will recognize how to perform any of these, or other, methods in accordance with the present invention.

In one embodiment, the methods of the invention are used in diagnostic methods. For example, the methods can be used to distinguish among different types of cancer; to monitor the effect of a treatment on a diseased cell; or to monitor the susceptibility of cells to treatment with an agent (e.g., drug) of interest, which can allow one to select a preferred drug regimen for a given patient, thereby providing individualized treatment for that patient.

Other uses of the methods of the invention are to prepare a database of profiles in response to various drugs, then test putative agents to determine if they are similar to or different from old ones in the profile; and to study mechanisms of disease and potential therapies. In particular the system described herein for characterizing signal transduction pathways involved in T-cell mediated conditions, can provide insights into the mechanisms, and possible treatments, for diseases or conditions such as autoimmune diseases, graft v. host disease, allergy, cancer and infectious diseases. Furthermore, the methods can be used in toxicity studies or to identify cross-reactions of certain drugs, or to study compounds developed by combinatorial chemistry.

Another aspect of the invention is a kit, comprising
(1) a plurality of (e.g., at least about three) recombinant constructs, each of which comprises an expression control sequence from a coordinated system of interest operatively linked to a reporter (The constructs may be in any suitable form, e.g., lyophilized or in an appropriate aqueous solution, such as a buffer);
(2) at least about three agents from a first set of agents that are known or predicted to act on at least one of said expression control sequences; and
(3) at least about three agents from a second set of agents, wherein said at least about three agents from said first and second sets of agents are suitable to be combined in an inter-set combinatorial fashion (the agents of the first and/or the second sets of agents may be in any suitable form, e.g., lyophilized or in an appropriate aqueous solution, such as a buffer; and the members of each set of agents may be packaged individually or in various combinations); and, optionally,
(4) an electroporation device suitable for electroporating said recombinant constructs into suitable cells; and/or
(5) instructions for how to detect the effects of the agents on the expression control sequences.

In another embodiment, the kit comprises
a) expression control sequences from genes involved in signal transduction pathways, e.g., signal transduction genes involved in T-cell stimulation (e.g., the promoter etc. sequences in column 1 of Table 1, from IL-2, CD28RE-TRE NFAT, AP-1, NFkB, CREB, UAS/p300 N-term, UAS/p300 FL), or Smad binding sites, Stat (1-6) binding sites, SP-1 binding sites, c-myc binding sites, ets binding sites, ATF-2 bindings sites, C/EBP binding sites, HIV-LTR, MMTV-LTR, HTLV-1-LTR, Erg-1 binding sites; gamma interferon activated sequence (GAS), GATA 1-3 binding sites, Oct-1,2 binding sites, LMO-1,2, P53 binding site, E2F-1,2 binding sites, ZBP 89 binding sites, or HSV-8 promoter),
b) a first set of agents that comprises stimulatory and/or regulatory agents, such as, e.g., mitogens, growth factors, and/or hormones that act on the expression control sequences of a) above, e.g., the agents in column 2 of Table 1, testosterone and analogues, estrogen and analogues, insulin, EGF (epidermal growth factor, NGF (nerve growth factors), Interleukins (1-15), Rantes family, TNF family (tumor necrosis factor), adrenalin, corticosteroids, human growth hormone, anabolic steroids, progestins, prolactin, thyroid hormones, pituitary hormones, parathyroid hormones, vaso-intestinal peptide, gastrin, all forms of (CSF's) colony stimulating factors, all forms of oral contraceptives (any of the preceding agents would be useful in translational clinical research), and
c) a second set of agents that comprises pharmaceutical agents, such as immunomodulatory agents, e.g., drugs that have been used in humans, such as the agents in column 3 of Table 1; or the immunomodulatory agents FK506, Pentoxfiline, Methotrexate, Dexamethasone, or rapamycin; or the following pharmacological agents, all of which modify cellular signaling pathways: anti-diarrheals, anti-hypertenson, anti-histamines, narcotic agents, anti-anxiolytic agents, anti-depressants, anti-metabolite agents, including over the counter drugs, herbal remedies, all oral and intravenous chemotherapeutic agents, new line chemotherapeutic agents, anti-angiogenesis agents, histone deacetylase inhibitors, active ingredients in food, caffeine, MSG (mono-sodium glutamate), all viral gene therapy agents including empty viral vectors.

Such kits have many uses, which will be evident to the skilled worker. For example, they can be used to characterize agents, such as putative therapeutic agents. That is, an agent of interest can be characterized by performing assays with the kit, and comparing the results to those obtained with known agents (or by comparison to a reference database of the invention). Such assays should be of commercial use, e.g., in high-throughput drug studies.

Other optional elements of a kit of the invention include suitable buffers, substrates, cofactors, inhibitors, and the like; a computer or computer-readable medium for storing and/or evaluating the assay results; logical instructions for practicing the methods described herein; logical instructions for analyzing and/or evaluating the assay results as generated by the methods herein; or packaging materials.

In a most preferred embodiment, methods of the invention relate to signal transduction pathways. To illustrate the power of the inventive method, studies are presented herein that characterize transcriptional regulation of the complex set of signal transduction pathways involved in the activation of T-cells. Aspects of this method are illustrated in Example I. To summarize those findings briefly:

Cellular behavior in response to changes in the cellular environment is controlled through extra-cellular events that are biochemically “transduced” at the cell membrane, and through a series of molecular signaling pathways converge in the nucleus to influence the combination of transcription factor binding sites that control the activation of targeted genes. Most of those promoter or regulatory regions of gene loci have a modular structure that is bound by two or more different transcriptional factors in a highly cooperative fashion. Accordingly, it is the nature of the surrounding regulatory elements or “promoter context” that combine to determine how genes are transcriptionally regulated. The present invention allows one to achieve a clear picture of the level of signal integration that occurs at these transcriptional targets.

Specifically, methods of the invention can be used to provide a “widescreen view” of the signal integration events at the level of transcriptional targets in live T-cells, using a high throughput approach. In Example I, eight different expression control sequences (e.g., promoter regions or isolated promoter elements, each known to be major site of transcriptional regulation during T-cell activation) are introduced, by simultaneous electroporation, into human T-cells (Jurkat T-cells). Each of the expression control sequences is operatively linked to a reporter—luciferase—and transcription under the control of the expression control sequence is detected as a function of luciferase activity. The transfected cells are then challenged with 16 different combinations of T-cell mitogens, and the transcriptional activation of each transcriptional target in the transfected population of cells is measured by standard procedures.

Multiple data sets are generated, each representing the activation profile of a single expression control sequence when challenged with 16 different combinations of mitogens. These activation profiles are then compared in the presence and absence of an array of 8 different immuno-modulatory drugs. Since there are 8 promoters in this particular study, 8 different data sets (raw response profiles) are generated. The total picture of these data can be described by a 16×64 matrix representing the stimulation of the 8 different promoters in a 64 dimensional space.

These generated multi-dimensional data can be stored in a database for future reference, but they are virtually impossible to describe graphically in physical space. Various multi-variate analytic techniques, such as principal component analysis (a dimension reduction using an Eigen vector to remap the original data points so that they can be displayed and compared in 3 or fewer dimensions), hierarchical clustering, unsupervised neural networks and ANOVA studies, are used to compare and contrast the profiles of the “mitogen responsiveness” of the transcriptional targets and the selective activation by the different T-cell mitogens and drugs.

As shown in Example I, this approach allows one to compare similarities and differences in the effects of mitogens and drugs on the responsiveness of promoter and promoters elements, providing information in a context that can be used to reveal or uncover previously unrecognized regulatory pathways, control points, and therapeutic targets. The methods of the invention provide a way to interrogate other complex combinatorial signaling systems, as well, and provide great insights into, i.a., the signal transduction language that cells use to control gene expression. Moreover, the methods provide a powerful platform through which combinatorial drug strategies can be evaluated in a pre-clinical setting. Other advantages of the methods are disclosed elsewhere herein.

Thus, a preferred embodiment of the invention is a method for generating multi-factorial biological response profiles, comprising
a) exposing each member of a plurality of expression control sequences, each of which is from a signal transduction gene (e.g., a signal transduction gene involved in the T-cell activation pathway), and/or is from a gene that is responsive to a signal transduction protein, independently, to
   at least about three stimuli from a first set of agents known to have an effect on the expression control sequences, wherein at least about two (e.g., at least about three) of the stimuli in the first set are combined in an intra-set combinatorial fashion, and to
   at least about three stimuli from a second set of agents, wherein at least about two (e.g., at least about three) of the stimuli in the second set are optionally combined in an intra-set combinatorial fashion,
   in an inter-set combinatorial fashion,
wherein each of the expression control sequences is operatively linked to a heterologous reporter in a recombinant construct, and
wherein each of the recombinant constructs is introduced into a cell that comprises one or more signal transduction genes, or genes that are responsive to signal transduction proteins (e.g., a T-cell (T-lymphocyte) or related cell (e.g., a primary T cell, a tumor, or an established T-cell line, such as Jurkat)); and all of the recombinant constructs are introduced into the cells simultaneously,
b) detecting the responses of said expression control sequences to said stimuli, and
c) generating a response profile for each of said expression control sequences.

In a preferred embodiment,
the plurality of expression control sequences comprise one or more of: the promoters etc. in column 1 of Table 1 (from, IL-2, CD28RE-TRE, NFAT, AP-1, NFkB, CREB, UAS/p300 N-term, UAS/p300 FL), Smad binding sites, Stat (1-6) binding sites, SP-1 binding sites, c-myc binding sites, ets binding sites, ATF-2 bindings sites, C/EBP binding sites, HIV-LTR, MMTV-LTR, HTLV-1-LTR, Erg-1 binding sites; gamma interferon activated sequence (GAS), GATA 1-3 binding sites, Oct-1,2 binding sites, LMO-1,2, P53 binding site, E2F-1,2 binding sites, ZBP 89 binding sites, or HSV-8 promoter,
the stimuli in the first set of agents comprise stimulatory and/or regulatory agents, such as mitogens, growth factors, hormones, or the like, and the stimuli in the second set of agents comprise pharmacological agents (such as immunomodulatory agents) known or expected to modify cellular signaling pathways.

In another embodiment,
the stimuli in the first set of agents comprise those listed in column 2 of Table 1, or testosterone and analogues, estrogen and analogues, insulin, EGF (epidermal growth factor, NGF (nerve growth factors), interleukins (1-15), Rantes family, TNF family (tumor necrosis factor), adrenalin, corticosteroids, human growth hormone, anabolic steroids, progestins, prolactin, thyroid hormones, pituitary hormones, parathyroid hormones, vaso-intestinal peptide, gastrin, all forms of (CSF's) colony stimulating factors, or all forms of oral contraceptives,
the stimuli in the second set of agents comprise those listed in column 3 of Table 1; or the following immunomodulatory agents: FK506, Pentoxfiline, Methotrexate, Dexamethasone, rapamycin; and/or the following pharmacological agents, all of which modify cellular signaling pathways: Anti-diarrheals, Anti-hypertenson, Anti-histamines, Narcotic agents, Anti-anxiolytic agents, Anti-depressants, Anti-metabolite agents, Including over the counter drugs, Herbal remedies, All oral and intravenous chemotherapeutic agents, New line chemotherapeutic agents, Anti-angiogenesis agents, Histone deacetylase inhibitors, Active ingredients in food, Caffeine, MSG (mono-sodium glutamate), all viral gene therapy agents including empty viral vectors, hallucinogenic drugs, neuroleptics and all sedatives,
and the recombinant constructs are electroporated into the cells.

The method may also further comprise
d) inputting the responses into a computer, thereby generating a database that comprises a raw profile for each of the expression control sequences,
e) processing the data base comprising the raw profiles with
   i) principal component analysis,
   ii) hierarchical clustering,
   iii) unsupervised neural networks, and/or
   v) ANOVA studies,
   or a combination thereof, and, optionally,
f) displaying the processed profiles.

Example II describes a high throughput method for nuclear run-on analysis. This procedure (and arrangements of oligonucleotides used to implement it) can be used independently, or in conjunction with the methods of the present invention.

One aspect of the invention is in a nuclear run-on method to characterize mRNA transcription from a genomic DNA template of interest, the improvement comprising hybridizing labeled nascent mRNAs transcribed from said genomic DNA template to an arrangement (e.g., an array) of at least about three addressable sets of oligonucleotide probes attached to a surface, wherein each of said sets of oligonucleotide probes is complementary to a different (e.g., non-overlapping) portion of the mRNA transcribed from said genomic DNA template. As a control, it is sometimes desirable to include one or more sets of probes that are complementary to an untranscribed portion of the genomic DNA (e.g., sequences upstream of the 5′ mRNA start site). Furthermore, a set of oligonucleotide probes corresponding to sequences at or near the 3′ terminus of an mRNA (e.g., corresponding the about 200 3′-most nucleotides) can allow an investigator to detect the presence of full-length transcripts.

In embodiments of this method, the labeled nascent mRNA is harvested at at least about two (e.g., at least about three or four) time points during transcription in the presence of a label; and samples from each of the time points are hybridized to the arrangement of probes.

Said arrangement of oligonucleotide probes comprises a plurality of sets of probes, preferably
a) first set of one or more oligonucleotides complementary to the about 200 nt upstream of the 5′ end of the transcribed mRNA. In a preferred embodiment, the set comprises at least about three separate oligonucleotides, e.g., contiguous oligonucleotides, or overlapping oligonucleotides that are each about 60-70 nt in length,
b) a second set of one or more oligonucleotides complementary to the first about 200 nt of the 5′ end of the transcribed mRNA. In a preferred embodiment, the set comprises at least about three separate oligonucleotides, e.g., contiguous oligonucleotides, or overlapping oligonucleotides that are each about 60-70 nt in length,
c) a third set of one or more oligonucleotides complementary to the second about 200 nt of the 5′ end of the transcribed mRNA. In a preferred embodiment, the set comprises at least about three separate oligonucleotides, e.g., contiguous oligonucleotides, or overlapping oligonucleotides that are each about 60-70 nt in length, and
d) a fourth set of one or more oligonucleotides complementary to the final about 200 nt of a full-length mRNA transcribed from said template. In a preferred embodiment, the set comprises at least about three separate oligonucleotides, e.g., contiguous oligonucleotides, or overlapping oligonucleotides that are each about 60-70 nt in length.

Of course, sets of oligonucleotides corresponding to (complementary to) sequences that are shorter or longer than 200 nt may be used. Furthermore, the size of the oligonucleotide probes within each set may be smaller, or larger, than about 60-70 nt. A skilled worker will recognize appropriate probes to use in the method.

Methods of attaching the sets of probes to an appropriate surface are conventional. For example, each set of at least about three oligonucleotides may be applied together to a surface (e.g., spotted together, for example on a glass slide); or each of the oligonucleotides may be independently attached to the surface, e.g., placed on different addressable locations on a gene chip.

In a preferred embodiment, the method is high throughput. One or more of the processes may be achieved robotically.

In one embodiment, sets of oligonucleotides corresponding to splice sites may be used, allowing an investigator to monitor splicing events.

In general, the movement of RNA polymerase molecules on a plurality (preferably a large number) of different DNA templates is monitored simultaneously. See, e.g., Example II, in which the transcription of four genes is monitored.

Methods to perform nuclear run-on assays are conventional. See, e.g., Groudine et al., (1981), Mol Cell Biol. 1(3), 281-8 and Marzluff et al. (1978), Methods Cell Biol. 19, 317-32.

Another aspect of the invention is an arrangement of at least about three addressable sets of oligonucleotide probes attached to a surface, for analyzing mRNA transcription from a DNA template of interest, comprising the sets of oligonucleotides as described above. Methods of designing and making suitable oligonucleotide probes, and attaching them to a suitable surface (e.g., attaching them covalently, or spotting them onto a surface like a glass slide), are conventional and well-known to those of skill in the art. For a discussion of some such methods, as well as factors that can be varied in designing and making surfaces comprising probes for high throughput assays, see U.S. Pat. No. 6,458,533.

Another embodiment of the invention relates, e.g., to a method for generating multi-factorial biological response profiles (including expression profiles), comprising
a) exposing each member of a plurality of biological entities, independently, to at least about three stimuli from a first set of stimuli, and to at least about three stimuli from a second set of stimuli, in an inter-set combinatorial fashion,
   i) wherein at least about two (e.g., at least about three) members of the stimuli in said first set of stimuli are, optionally, combined in an intra-set combinatorial fashion, but none of the members of the stimuli in the second set of stimuli are combined in an intra-set combinatorial fashion, or
ii) wherein at least about two (e.g., at least about three) members of the stimuli in said second set of stimuli are, optionally, combined in an intra-set combinatorial fashion, but none of the members of the stimuli in the first set of stimuli are combined in an intra-set combinatorial fashion, or
iii) wherein
at least about two (e.g., at least about three) members of the stimuli in said first set of stimuli are, optionally, combined in an intra-set combinatorial fashion, and
at least about two (e.g., at least about three) members of the stimuli in said second set of stimuli are, optionally, combined in an intra-set combinatorial fashion, and
the members of the first set and the second set of stimuli represent different categories of stimuli,
b) detecting the responses of said biological entities to said stimuli, and
c) generating a response profile for each of said biological entities.

As used herein, the term “biological entity” includes any entity from a biological source that can respond to a stimulus. Biological entities range, e.g., from sub-domains within transcriptional promoters to intact cells or tissues. One type of biological entity is an expression control sequence, preferably operatively linked to a reporter, as discussed elsewhere herein.

The biological entities may be studied in vivo. For example, they may be cells, or expression control sequences that have been introduced into cells. In a preferred embodiment, all of the biological entities are studied in vivo. Alternatively, the biological entities may be studied in vitro. For example, one may expose to stimuli nuclei that have been extracted from cells, or other forms of cell extracts. Nuclear run-on assays are examples of such in vitro assays.

An expression control sequence studied by the methods of the invention may be in a form in which it is operatively linked to the coding sequences it naturally regulates. In such a case, the gene may be studied in the cell in which is naturally occurs; or it may be isolated (e.g., cloned into an expression vector) and introduced into a heterologous cell. The term “isolated,” when referring to a nucleic acid or protein as used herein, means in a form that is not found in its original environment or in nature, e.g., more concentrated, more purified, separated from at least one other component with which it is naturally associated, in a buffer, in a dry form awaiting reconstitution, etc.

Samples to be analyzed by a method of the invention can be obtained from recombinant constructs (e.g., as discussed above), or the samples can be obtained (or derived) from any of a variety of sources that will be evident to the skilled worker. These sources include, e.g., cells (either within a plant or animal, or taken directly from a plant or animal, or a cell maintained in culture or from a cultured cell line); lysed cells (including lysate fractions) or cell extracts; supernatants of cultured cells (which include, i.a., secreted proteins); organs; tissues; or bodily fluids (e.g., lymph, urine, blood, sputum); or the like. Samples may be obtained from a prokaryote, plant or animal (e.g., a human or non-human primate, or a domestic, farm, or laboratory animal, such as a horse, dog, cat, bird, ferret, cow, pig, sheep, goat, rat, mouse, rabbit, guinea pig, fish, or frog). In vitro reactions can include a molecule derived from a cell or cellular material (e.g., a polypeptide or nucleic acid molecule); or can be an experimental reaction mixture (e.g., containing a buffer and salts, substrates, and/or any other molecules needed to carry out an assay) which is to be assayed or analyzed according to the methods of the invention.

In the foregoing and in the following examples, all temperatures are set forth in uncorrected degrees Celsius; and, unless otherwise indicated, all parts and percentages are by weight.

### EXAMPLES

### I. A Study of Regulatory Elements Involved in T-Cell Activation

A. Summary of the Experimental System

A human acute lymphoblastic T-cell leukemia cell line (Jurkat) was individually electroporated with 5 ug of a luciferase reporter plasmids driven by the interleukin 2 (IL-2) promoter, the NFAT element of the IL-2 promoter, the CD28RE-AP1 element of the IL-2 promoter, a consensus AP-1 element, a consensus NF-kappa B element, a consensus CRE element and the co-transfection of a Gal4 binding site or upstream activation sequence (UAS) driving a luciferase reporter in combination with either a full length Gal4-p300 fusion (FL) or a N-terminal domain fusion of p300 (amino acids 1-743). Electroporation was performed in a 96 well format with a BTX 96-well gold electrode. Cells were electroporated at 10 millions cells per well with each experiment repeated in triplicates. Following electroporation cells were stimulated with various combination of T-cell mitogens (see Table 1). Cells were harvested 5 hours after stimulation and assayed for luciferase activity by a 96 well luminometer. Data were normalized first for fold activation for each experiment and then inputted into an activation matrix where the data were normalized to percent maximum stimulation. The multidimensional data were then remapped by principal component analysis into a 3 dimensional statistical space where the unit of distance along each axis has been reformatted from fold activation to an extracted variance or statistical distance. Therefore distance between points in 3D space represents the similarity or difference between the points (or mitogens) in terms of a “statistical summation” of how they activated the 8 different promoter elements.

TABLE I
The 8 promoter elements (left) were stimulated with
16 different combinations of T-cell mitogens (center),
in the presence and absence of 8 different drugs.
Pharmacological profiling of the transcriptional
targeting by T-cell mitogens

| | | T-cell mitogens | | |
|---|---|---|---|---|
| Regulatory elements | | (16 combinations) | | Drugs/Ligands |
| IL-2 | vs | Unstim | vs | CSA |
| CD28RE-TRE | | PMA | | SB203580 |
| NFAT | | Ionomycin | | Wortmanin |
| AP-1 | | PHA | | PD98059 |
| NFkB | | Anti-CD3 | | Forskolin |
| CREB | | Anti-CD28 | | Rottlerin |
| UAS/p300 N-term | | | | TGF-beta |
| UAS/p300 FL | | | | IGF-1 |

B. Analysis by Principal Component Analysis (PCA)

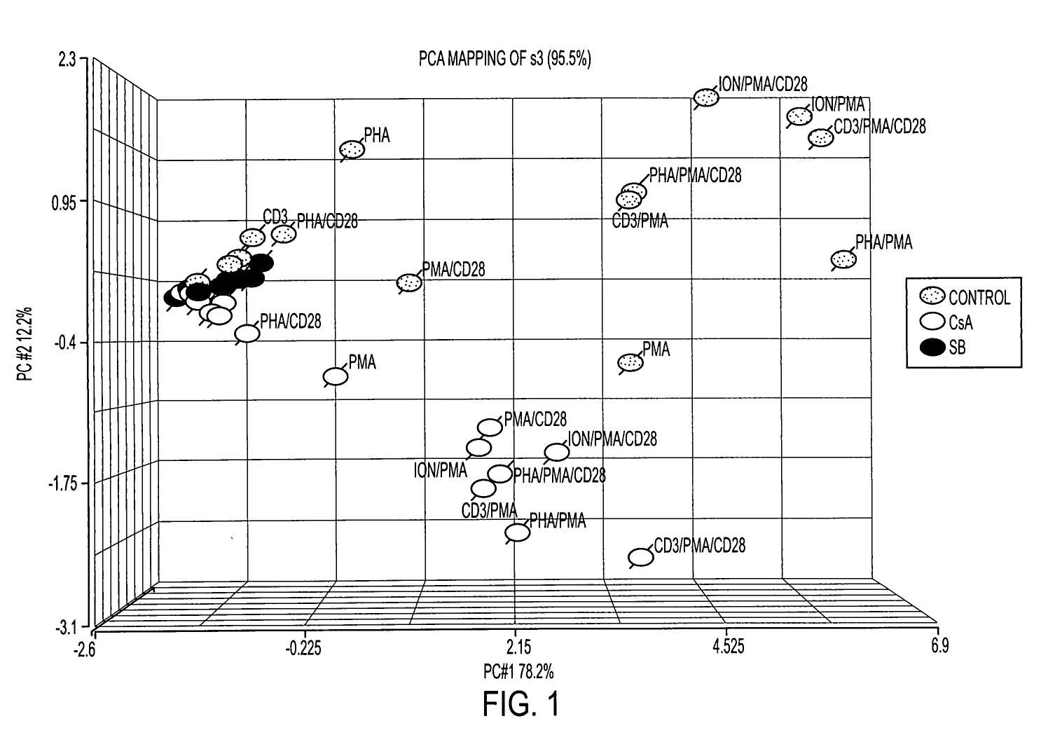

FIG. 1 shows a principal component analysis of 16 different mitogen combinations in the presence of 3 different drug conditions (control, cyclosporine A, and SB203580). This experiment generates data describing how the 16 combinations of mitogens activated 8 different promoters in the presence and absence of cyclosporine A (CSA) and SB203580 (SB). Here the 16×3 combinations of mitogens and drugs are dispersed as 48 data points in 3 dimensions. These 3 dimensions are a mathematical composite of the original promoter axes derived by an eigen vector calculation with a subsequent remapping of the data points. When the mitogen combinations are color coded according to what drug was added, a clear spatial segregation of the drug treated groups becomes evident. In this way, the analysis provides a very precise means of determining the similarities and differences in how specific drugs target multiple mitogen-dependent signaling pathways in activated T-cells.

C. Analysis by Hierarchical Clustering

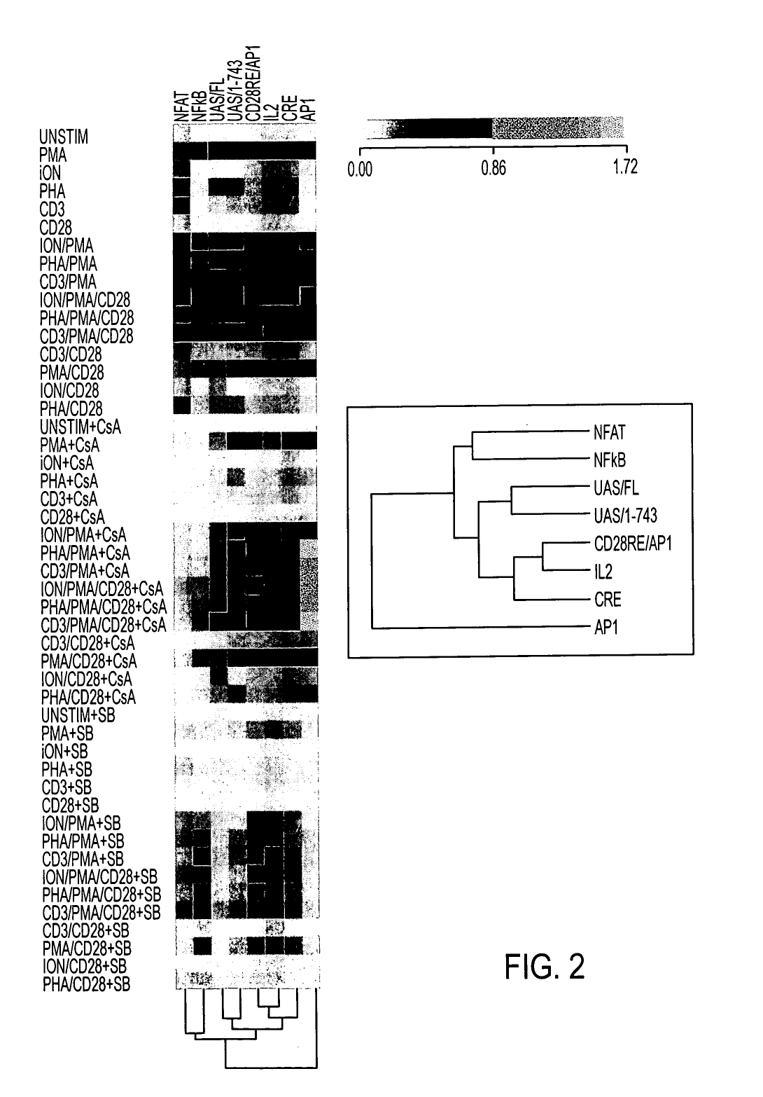

Using another computational method referred to as hierarchical clustering, the data generated in FIG. 1 can be analyzed to compare the mitogen and drug responsiveness of the different promoter elements. As shown in FIG. 2, a comparison of the 8 promoter elements by their responses to the 48 combinations of drugs and mitogens separates them into two major classes. One of them shows the AP-1 response element as segregated in a class by itself. A clear distinguishing feature is that the AP-1 is upregulated in the presence of cyclosporine A. The other class is divided into two subclasses; one where NFAT and the NF-kB elements are grouped together, and another that contains sub-groupings of the remaining elements.

D. Analysis by Self Organizing Maps (SOM)

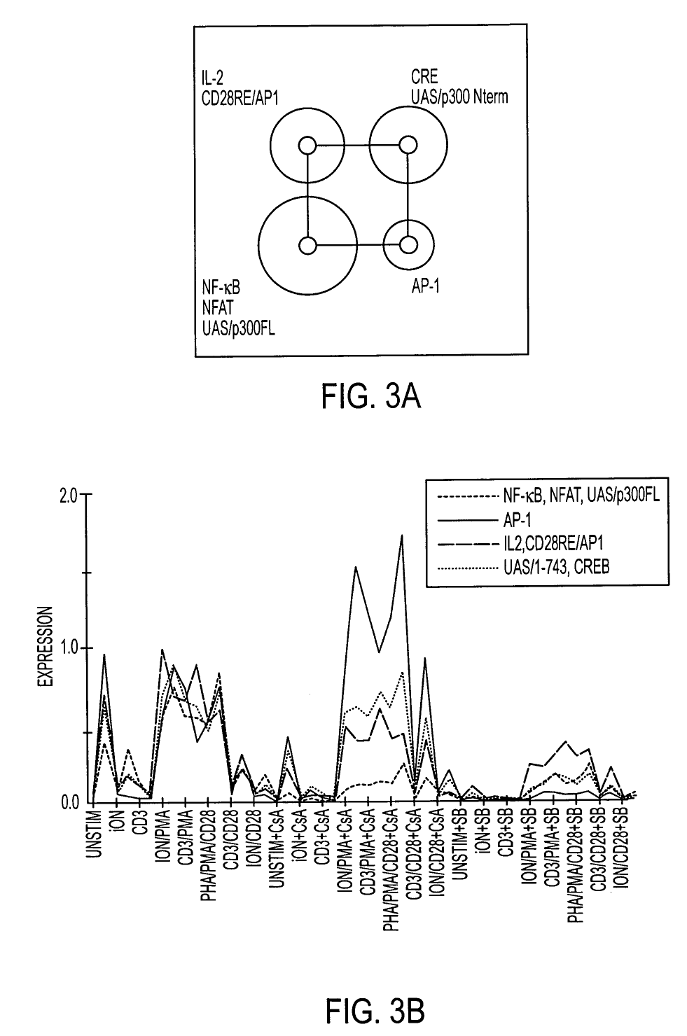

Similar results are obtained with added information content by a form of unsupervised neural networks analysis referred to as self organizing maps. Self organizing maps (SOM) is another class assignment method that attempts to cluster groups of variables into a pre-determined number of classes. As shown in FIG. 3, where the promoter elements were grouped into 4 maximum categories, once again, AP-1 was placed in a class by itself and NFAT and NF-kB were grouped together. In addition, SOM generates a “centroid” plot that represents a composite profile of the overall mitogen and drug responsiveness of each class.

E. Interpretation of the Statistical Analysis

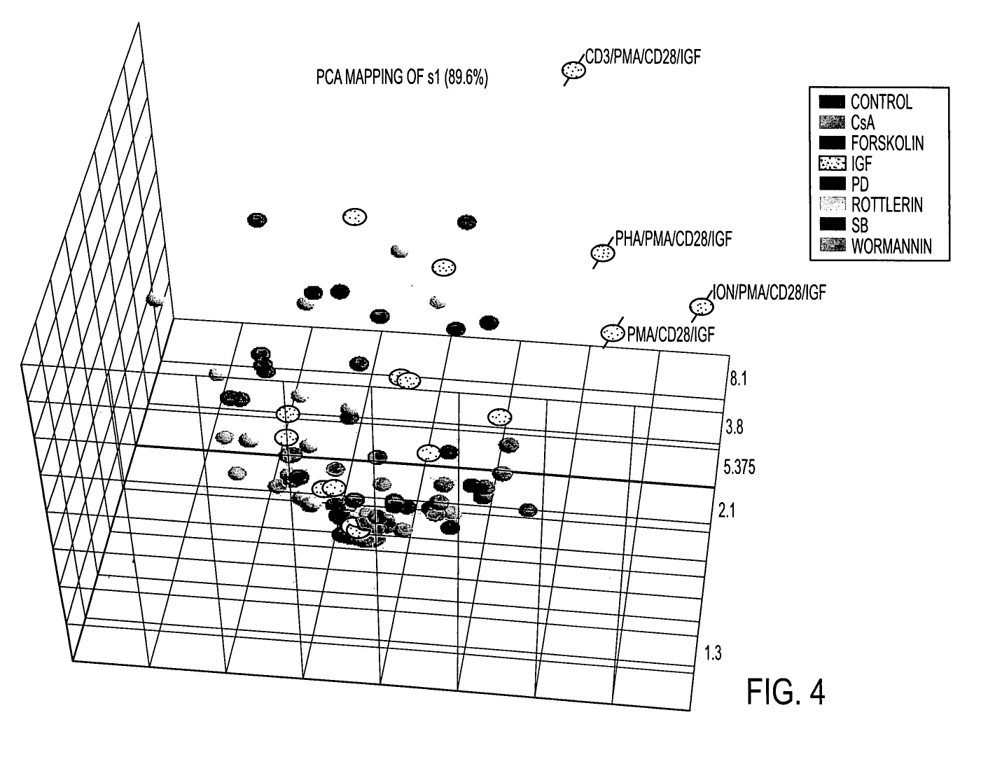

FIG. 4 shows a principal component analysis of the 16 mitogen combinations in the presence of 8 different drug/hormone conditions. The condensation of this high dimension data into 3 dimensions rapidly reveals 4 major outliers. Each one of the outliers occurred in the presence of insulin-like growth factor one (IGF-1) (blue). IGF-1 is a peptide growth factor that has been strongly implicated in a variety of cancers and whose serum levels in different animal models show a strong correlation with increased cancer risk. These data show that IGF synergies with anti-CD28 stimulation in activated T-cells to upregulate the NF-kappa B pathway. Further analysis indicates that the four outliers require the presence of both IGF-1 stimulation and co-stimulation with antibodies that trigger signaling through the CD28 co-receptor molecule.

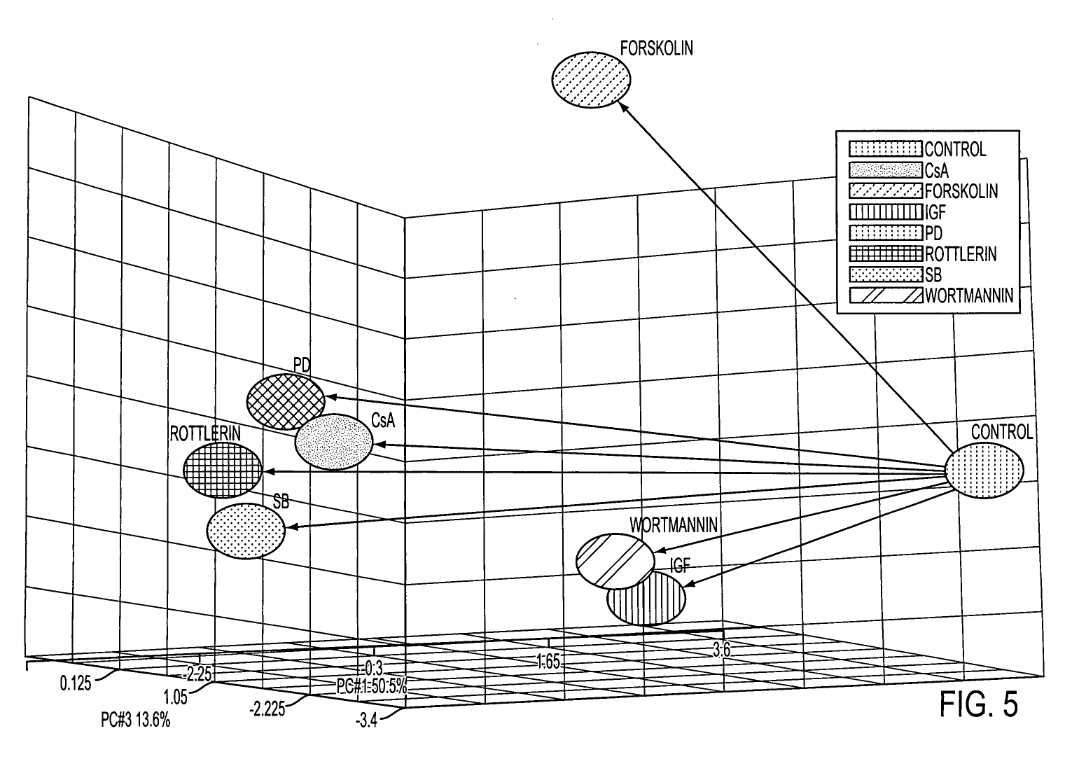

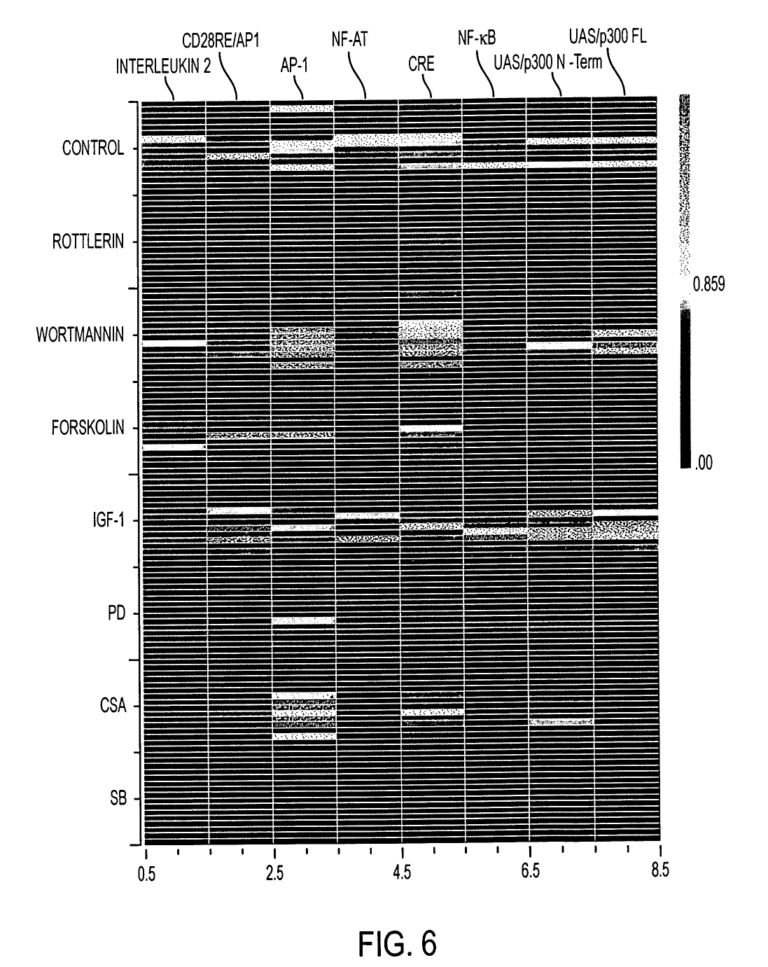

FIG. 4 shows that principal component analysis of the multi-dimensional data can reveal the presence of previously unrecognized control points during T-cell activation. The labeled outliers (blue) in FIG. 4 indicate that the combined presence of anti-CD28 co-receptor stimulation and IGF-1 produces a strong influence on the profile. Refined examination of the data reveals that this signaling occurs predominantly through a targeting of the NF-kappa B family of transcription factor (see FIG. 6). As shown in FIG. 5, there is a significant degree of similarity between the pathways targeted by the P13 kinase inhibitor, wortmannin and IGF-1. Forskolin (brown) shows a targeting profile that is completely different from the other conditions, whereas IGF (blue) and wortmannin (yellow) appear to target the same pathway. Indeed, it is known that IGF-1 receptor signaling in many cells is controlled by or “upstream of” P13 kinase, a multifunction kinase that increases the level of lipid secondary messengers. The combined interpretation of these data suggests that this pathway is operational in T-cells and directly up-regulates NF-kappa B dependent genes.

These findings suggest IGF-1 as a treatment and diagnostic agent in malignancies, immuno-deficiencies and immunosuppression. For example, samples of lymphoid tissue can be screened for IGF-1 concentrations by conventional histochemistry techniques. Increased IGF-1 levels are associated with an increased risk for cancer. Levels can also be assessed in other metabolic diseases, including diabetes. Furthermore, age-related changes in concentrations of IGF-1 in lymphoid tissue can be detected. The effect of chemotherapeutic and hormone treatment on the levels of IGF-1 in lymphoid tissues can be monitored. Therefore, one aspect of the invention is a method to screen for the presence of a malignancy, immunodeficiency, or autoimmunity (including autoimmune damage to a tissue, such as, e.g., kidney, pancreas, brain, gut or liver) in a patient in need thereof, comprising assaying lymphoid tissue of said patient for the presence of an increased level of IGF-1 in the tissue compared to the level in a normal lymphoid tissue. Procedures for performing such analysis, such as immunohistochemistry, in situ hybridization, or RT-PCR techniques, are conventional.

### II. Nuclear Run-On Analysis

Another embodiment of the invention involves measurement of the number of engaged nuclear RNA polymerases, in vivo, at certain time points following a signal transduction event, e.g. in T-cells. This is accomplished by conventional nuclear run-on (NRO) analysis, in which nuclei from cells treated for specific times, under selected conditions, are isolated via methods that preserves the number of RNA polymerases engaged at the endogenous genes. Free nucleotides that are labeled with isotope or by a means that allows fluorescent detection are added to the nuclei. This allows the polymerases at each endogenous gene in the nuclei to continue to elongate, thus incorporating the labeled nucleotides. This labeling provides a way of direct detection of only newly made or “nascent” RNA by subsequent hybridization onto synthetic nuclei acid targets. Methods of performing such nuclear run-on analysis are conventional and are described, e.g., in Groudine et al., (1981), Mol Cell Biol. 1(3), 281-8 and Marzluff et al. (1978), Methods Cell Biol. 19, 317-32.

Conventional modes of detection of such nuclear run-on products involve hybridization using known complementary (or anti-sense) PCR products or oligonucleotides adsorbed to a membrane substrate, such as nylon or nitrocellulose. In this manner, the amount of newly synthesized nuclear RNA produced from a single gene can be assessed. One embodiment of the present invention takes advantage of the miniaturization methods of microarray technology, which allow for the simultaneous examination of as many as hundreds to thousands of genes. Nuclear run-on analysis, using either microarray technology or smaller numbers of “sets” of probes, may be used in conjunction with the inventive methods. Through this approach, as many as hundreds to thousands of genes can be analyzed under, e.g., multiple mitogen and drug combinations, thus adding multiple dimensions to the profile of the transcriptional activation of the endogenous genes.

The samples may be derived from naturally endogenous genes in a cell. Alternatively, episomal vectors that contain expression control sequences of interest may be introduced into a cell and allowed to form a chromatin-like structure; or vectors containing expression control sequences of interest may be allowed to integrate into the host genome, thereby attaining a chromatin-like structure.

In a preferred embodiment, arrays of “sets” of synthetic oligonucleotides are used. Each “set” corresponds to a different (non-overlapping) about 200 nt sequence of the mRNA or genomic template. Preferably, the 200-mer sequences represent sequential increments along the newly synthesized nascent mRNA. That is, the sets of oligonucleotides corresponding to these 200-mers serve as “tiles.” Each “set” may consist of a single oligonucleotide (e.g. an oligonucleotide of about 200 nt), or it may comprise about 3-6 smaller oligonucleotides (e.g., oligonucleotides of about 60-70 nts) that cover the 200 nt sequence. These smaller oligonucleotides may be contiguous, or they may be at least partially overlapping. A more thorough discussion of the oligonucleotides that may be used to detect nuclear run-ons is found elsewhere herein.

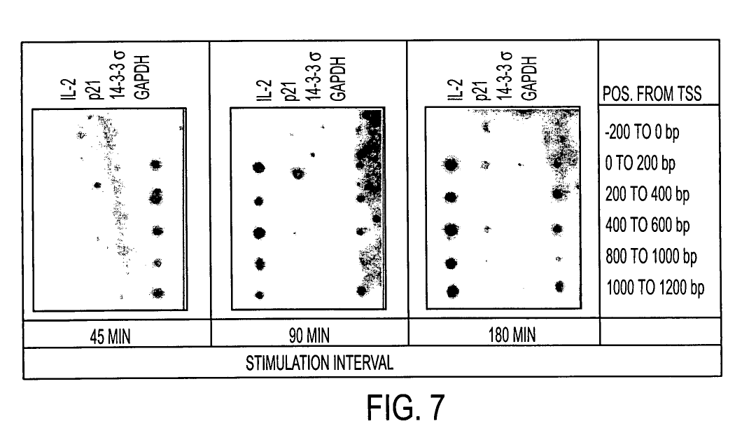

The oligonucleotides can be spotted onto any suitable surface, such as the glass slides shown in FIG. 7; or they can be attached to suitable surfaces (e.g., to form a “gene chip”).

This arrangement of oligonucleotide “tiling” gives an impression of how signal transduction events change the distribution, rate and number of engaged polymerases along the length of the specific genes in a stimulated population of cells. The tiling allows each of these to be measured with reference to the start of transcription. This procedure allows an extension of the application of the multi-dimensional molecular analysis of the invention to include an examination of the effect of signaling cascades on, e.g., peri- and post-transcriptional events, including elongation, kinetic loading of polymerases and RNA splicing.

### III. Proteomic Analysis

High throughput proteomic analysis that utilizes a rapid immuno-characterization technique capable of screening for translation (the presence and/or amount of protein) or for specific post-translational modifications of protein, allows for making real-time correlations between transcriptional activation and changes in the proteome during biological processes, e.g., during T-cell activation.

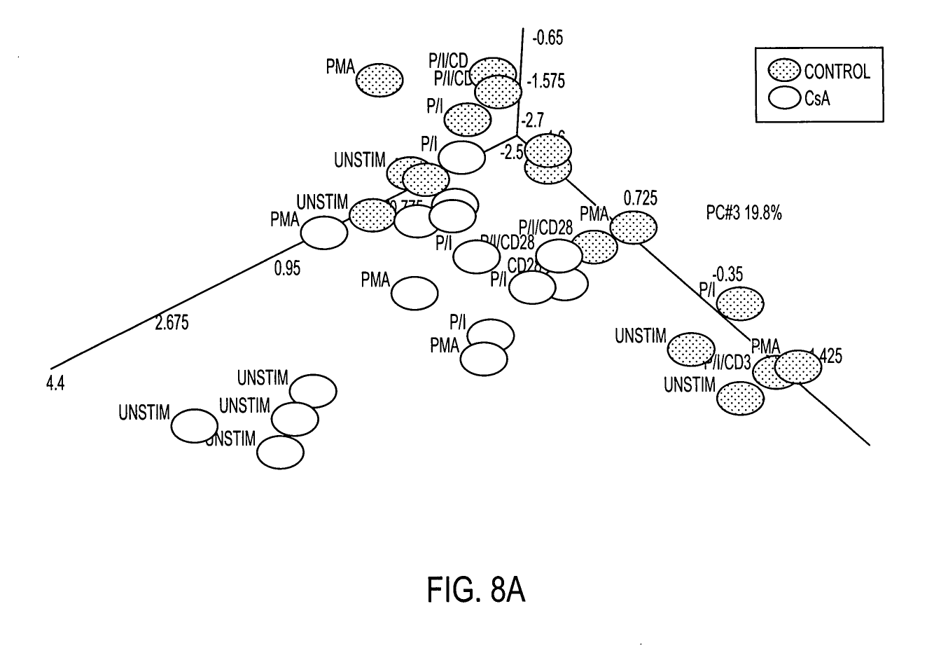

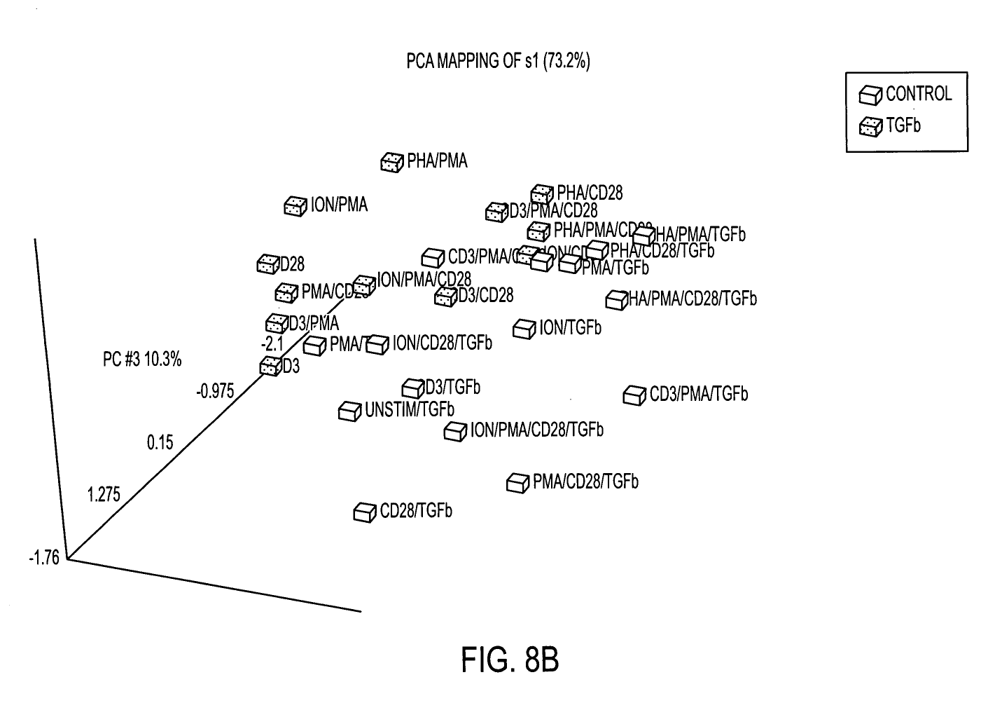

A molecular compartment that is dynamically changed during signal transduction is the proteome. We have adapted our high throughput electroporation methods to allow sampling of the cellular lysates, after, e.g., drug and mitogen treatment, for analysis of protein levels and/or post-translational modifications. This involves using a layered membrane approach to make multiple replicas of protein lysates on nitrocellulose membranes. For a further discussion of this approach, in general, see US published application 20020012920. In the present embodiment, the membranes are loaded with the samples in the same format (e.g., a 96-well format) used for the electroporation, so each sample is already scored and correlated with a transcriptional output. The spotted membranes (e.g., each with about 96 spots) are then probed with antibodies to specific proteins and/or protein modifications. This adds new proteomic dimensions to the transcriptional analysis (see FIG. 8). These data can be directly incorporated in the analysis as discussed elsewhere herein, as additional dimensions of analysis for PCA, for self-organizing maps, or hierarchical clustering. In the addition, the data analysis can stand on its own as a separate proteomic approach to illustrate the effects of mitogens or drugs on the T-cell proteome (FIG. 8).

### IV. Analysis of Kinetic Formation of Protein-DNA Complexes

An embodiment of the invention involves profiling the kinetic formation of protein-DNA complexes in the nucleus by combining standard chromatin precipitation analysis with microarray technology. A suitable DNA to use for such analyses is DNA introduced on a vector, such as an episomal vector, which is allowed to form a chromatin-like structure (e.g., following one cell cycle). By this approach, protein-DNA complexes are captured in vivo by chemical cross-linking. The complexes are then deproteinated and the isolated DNA is amplified. Fluorescent nucleotides are incorporated by subsequent rounds of DNA amplification. The amplified and labeled DNA is then hybridized to known target sequences spotted (or otherwise attached to a surface) to form an array. In this case the spotted sequences are PCR products representing known genomic segments of the promoters of various genes. This assay is performed at various time points following multiple stimuli, and immuno-precipitation with various antibodies against different transcriptional regulators. Thus a large set of multi-dimensional data is generated with dimensions that include specific DNA-associated protein, time points and mode of stimulation and drug treatment. Once again, these transcriptional profiles can be assessed by the computational approaches outlined elsewhere herein.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure of all applications, patents and publications, cited above and below and in the figures are hereby incorporated by reference. U.S. Provisional Application 60/461,410, filed Apr. 10, 2003, is also incorporated by reference herein in its entirety.

References

| | |
|---|---|
| US published patent applications | U.S. Pat. Nos. |

| | | |
|---|---|---|
| 20020164594 | 20020155420 | 6,263,287 |
| 20020064788 | 20030059818 | 6,203,987 |
| 20020155420 | 20020115070 | 6,468,476 |
| 20020012920 | 20020137077 | |
| 20020178150 | | |

1-76. (canceled)

77. A method for generating and analyzing multi-factorial biological response profiles, comprising
a) exposing each member of a plurality of expression control sequences, each of which is operatively linked to a heterologous reporter sequence, independently, to at least about three stimuli from a first set of stimuli, wherein at least about two of the stimuli in said first set of stimuli are, optionally, combined in an intra-set combinatorial fashion,
b) detecting a first category of responses of said expression control sequences to said stimuli, and
c) generating a response profile for each of said expression control sequences.

78. The method of claim 77, wherein at least about two of the stimuli in said first set of stimuli are combined in an intra-set combinatorial fashion.

79. The method of claim 77, further comprising
d) exposing each of said members of the plurality of expression control sequences, independently, to one or more additional sets of stimuli, optionally wherein at least about two of the stimuli in each of said additional sets of stimuli are combined in an intra-set combinatorial fashion,
in an inter-set combinatorial fashion with set first set of stimuli,
e) detecting the first category of responses of said expression control sequences to the stimuli in d), and
f) generating a response profile for each of said expression control sequences, which includes the responses detected in b) and in e).

80. The method of claim 79, wherein at least about two of the stimuli in each of said additional sets of stimuli are combined in an intra-set combinatorial fashion.

81. The method of claim 79, wherein said members of the plurality of expression control sequences are exposed, independently, to one additional set of stimuli.

82. The method of claim 77, further comprising detecting one or more different categories of responses of said expression control sequences in a) to said stimuli, and combining those responses with the responses detected in b) to generate a response profile for each of said expression control sequences.

83. The method of claim 79, further comprising detecting one or more different categories of responses of said expression control sequences in a) to said stimuli, and combining those responses with the responses detected in e) to generate a response profile for each of said expression control sequences.

84. The method of claim 77, wherein the response of the expression control sequences to the stimuli is one or more of the following categories:
a) levels of RNA produced in response to the stimuli,
b) levels of proteins translated from said RNA,
c) levels of post-translational protein modification,
d) movement of an RNA polymerase molecule along a DNA template (as determined by nuclear run-on analysis) in response to the stimuli, and/or
e) the formation of protein-DNA complexes (as determined by kinetic analysis) in response to the stimuli, and/or
f) changes in lipid membrane composition,
h) or a combination thereof.

85. The method of claim 84, further wherein the response of the expression control sequences to the stimuli is
i) responses detected at one or more different time points following the exposure of each of said expression control sequences to one of more of the stimuli in a) through h), and/or
j) responses to one or more different amounts (concentrations) or one of more of the stimuli.

86. The method of claim 79, wherein the stimuli in the first set and the stimuli in each of the additional sets represent different categories of stimuli.

87. The method of claim 86, wherein the different categories are:
a) agents that act at the surface of a cell vs. agents that function within a cell,
b) agents that exhibit different mechanisms of action,
c) agents that have different chemical structures vs. agents within a particular chemical class that differ from one another,
d) agents that are produced within a cell vs. agents that are introduced directly into a cell,
e) agents having a known mechanism of action on an expression control sequence vs. agents not having a known mechanism,
f) agents having a known effect on the expression control sequence vs. test agents,
g) agents known to have an effect on at least one of the expression control sequences vs. agents not known to have an effect on any of those expression control sequences,
h) naturally occurring agents vs. artificially generated molecules, and/or
i) physical agents vs. environmental stimuli, or
j) a combination thereof.

88. The method of claim 77, wherein the response profiles are raw profiles, and generating the raw profiles comprises inputting the responses into a database, thereby generating a database which comprises a raw profile for each of the expression control sequences.

89. The method of claim 77, wherein the response profiles are processed profiles, and generating the processed profiles comprises
inputting the responses into a database, thereby generating a database which comprises a raw profile for each of the expression control sequences, and
processing the data base comprising the raw profiles with a multivariate statistical method.

90. The method of claim 89, wherein the multivariate statistical method is
i) principal component analysis,
ii) hierarchical clustering,
iii) unsupervised neural networks, and/or
iv) ANOVA studies,
or a combination thereof.

91. The method of claim 89, wherein the responses are inputted into a computer.

92. The method of claim 89, further comprising displaying the processed profiles.

93. The method of claim 77, which is a high throughput method.

94. A method of claim 77, which is
a) a method for determining the type of an unknown stimulus,
b) a method for identifying/characterizing a modulatory or co-modulatory agent,
c) a method for identifying the cellular pathway affected by an agent,
d) a method for determining whether a drug candidate has an activity similar to a known drug,
e) a method for identifying a regulatory pathway, control point or therapeutic target,
f) a method for studying a combinatorial drug strategy in a pre-clinical setting,
g) a method for identifying a cell or organism that is sensitive or resistant to a drug composition,
h) a method for determining if a sample is susceptible to, or likely to benefit from, a particular treatment, or
i) a method for determining if an agent is toxic.

95. The method of claim 77, wherein the plurality of expression control sequences exhibit coordinated biological activity.

96. A computer-implemented method for generating and analyzing multi-factorial biological response profiles, comprising
a) exposing each member of a plurality of expression control sequences, each of which is operatively linked to a heterologous expression control sequence, independently, to
at least about three stimuli from a first set of stimuli, wherein at least about two (e.g., at least about three) of the stimuli in said first set of stimuli are combined in an intra-set combinatorial fashion, and to
at least about three stimuli from a second set of stimuli, wherein at least about two of the stimuli in said second set of stimuli are optionally combined in an intra-set combinatorial fashion,
in an inter-set combinatorial fashion,
b) detecting and inputting into a computer responses of said expression control sequences to said stimuli, thereby generating a database which comprises a raw profile for each of the expression control sequences,
c) processing the data base comprising the raw profiles with
i) principal component analysis,
ii) hierarchical clustering,
iii) unsupervised neural networks, and/or
v) ANOVA studies,
or a combination thereof, and, optionally,
d) displaying the processed profiles.

97. A kit, comprising
(1) a plurality of recombinant constructs, each of which comprises an expression control sequence from a coordinated system of interest operatively linked to a reporter,
(2) at least three about agents from a first set of agents that are known or predicted to act on at least one of said expression control sequences, and
(3) at least about three agents from a second set of agents,
wherein said at least about three agents from said first and second sets of agents are combined in an inter-set combinatorial fashion
and, optionally,
(3) an electroporation device suitable for electroporating said recombinant constructs into suitable cells; and/or
(4) instructions for how to detect the effects of the agents on the expression control sequences.

98. A computer system for generating and analyzing multi-factorial biological response profiles, comprising
a) means for inputting responses into a database, wherein said responses are generated by
i) exposing each member of a plurality of expression control sequences, each of which is operatively linked to a heterologous expression control sequence, independently, to
at least about three stimuli from a first set of stimuli, wherein at least two of the stimuli in said first set of stimuli are combined in an intra-set combinatorial fashion, and to
at least about three stimuli from a second set of stimuli, wherein at least about two of the stimuli in said second set of stimuli are optionally combined in an intra-set combinatorial fashion,
in an inter-set combinatorial fashion, and
ii) detecting the responses of said biological entities to said stimuli;
b) means for analyzing said inputted responses; and, optionally,
c) means for displaying the analyzed responses.

99. A computer-readable storage medium storing computer-readable program code for causing a computer to perform the following steps:
a) retrieving responses generated by
i) exposing each member of a plurality of expression control sequences, each of which is operatively linked to a heterologous expression control sequence, independently, to
at least about three stimuli from a first set of stimuli, wherein at least two (e.g., at least about three) of the stimuli in said first set of stimuli are combined in an intra-set combinatorial fashion, and to
at least about three stimuli from a second set of stimuli, wherein at least about two (e.g., at least about three) of the stimuli in said second set of stimuli are optionally combined in an intra-set combinatorial fashion,
in an inter-set combinatorial fashion, and
ii) detecting the responses of said biological entities to said stimuli,
b) processing the retrieved responses with a multivariate statistical method and, optionally,
c) displaying the processed responses.
